(19) United States
(12) Patent Application Publication
(10) Pub. No.: US 2023/0234989 A1
(43) Pub. Date: Jul. 27, 2023

(54) NOVEL SIGNAL PEPTIDES GENERATED BY ATTENTION-BASED NEURAL NETWORKS

(71) Applicants: California Institute of Technology, Pasadena, CA (US); BASF SE, Ludwigshafen (DE)

(72) Inventors: Michael LISZKA, San Diego (US); Alina BATZILLA, San Diego (US); Zachary WU, Pasadena (US); Frances ARNOLD, Pasadena (US)

(21) Appl. No.: 18/008,033

(22) PCT Filed: Jun. 4, 2021
(86) PCT No.: PCT/US2021/035968

§ 371 Date: Dec. 2, 2022

Related U.S. Application Data
(60) Provisional application No. 63/034,788, filed on Jun. 4, 2020.

(51) Int. Cl. C07K 14/00; G16B 25/10; G06N 20/00; C12N 9/26; C12N 9/24

(52) U.S. Cl. CPC C07K 14/00; G16B 25/10; G06N 20/00; C12N 9/2411; C12N 9/248; C12Y 302/01001; C12Y 302/01008; C07K 2319/02

(57) ABSTRACT

The disclosure provides for artificial signal peptides generated by systems and methods utilizing deep learning.

### STATEMENT OF FEDERAL GOVERNMENT SUPPORT

This invention was made with government support under Grant No. CBET-1937902 awarded by the National Science Foundation. The government has certain rights in the invention.

### FIELD OF TECHNOLOGY

The present disclosure relates to the field of biotechnology, and, more specifically, to an artificial signal peptide (“SP”) generated by systems and methods utilizing deep learning.

### BACKGROUND

For cells to function, proteins must be targeted to their proper locations. To direct a protein (e.g., to an intracellular compartment or organelle, or for secretion), organisms often encode instructions in a leading short peptide sequence (typically 15-30 amino acids) called an SP. SPs have been engineered for a variety of industrial and therapeutic purposes, including increased export for recombinant protein production and increasing the therapeutic levels of proteins secreted from industrial production hosts.

Due to the utility and ubiquity of protein secretion pathways, a significant amount of research has focused on identifying SPs in natural protein sequences. Conventionally, machine learning has been used to analyze an input enzyme sequence and classify the portion of the sequence that is the SP. While this allows for the identification of naturally-occurring SP sequences, generating a new SP sequence and validating the functionality of the generated SP sequence in vivo has yet to be performed.

Given a desired protein to target to an intracellular compartment or organelle, or for secretion, there is no universally-optimal directing SP and there is no reliable method for generating a SP with measurable activity. Instead, libraries of naturally-occurring SP sequences from the host organism or phylogenetically-related organisms are tested for each new protein sorting or secretion target. While researchers have attempted to generalize the understanding of SP-protein pairs by developing general SP design guidelines, those guidelines are heuristics at best and are limited to modifying existing SPs, not designing new ones.

### SUMMARY OF VARIOUS ASPECTS OF THE INVENTION

In one aspect, the present disclosure relates to artificially generated peptide sequences. The artificially generated peptide sequence may be an SP or a protein comprising the SP. In some embodiments, the SPs are used to express functional proteins in a host, such as a gram-negative bacteria. In other embodiments, the SP may be a peptide sequence having a length of 4 to 65 amino acids.

In other aspects, the present disclosure relates to artificial peptide sequences having an amino acid sequence selected from SEQ ID Nos: 1-164. In some aspects, the present disclosure relates to peptide sequences comprising an amino acid sequence selected from SEQ ID Nos: 1-164. In other aspects, the present disclosure relates to protein sequences comprising a SP conjugated to an amino acid sequence of a mature enzyme, wherein the SP is selected from SEQ ID Nos: 1-164. In some embodiments, the mature enzyme is an enzyme expressed in a gram negative bacteria, preferably in the genus Bacillus, most preferably a Bacillus subtilis. In still further embodiments, the mature enzyme is an amylase, dehalogenase, lipase, protease, or xylanase.

In some aspects, the present disclosure relates to artificial peptide sequences comprising an amino acid sequence that is a variant of any one of SEQ ID Nos: 1-164. In some aspects, a variant is a truncated form of any one of SEQ ID Nos: 1-164 (e.g., any 10, 11, 12, 13, 14, 15, 16, 17, 18, 19, 20, or >20 consecutive amino acids present in at least one of these sequences). In some aspects, the variant is a sequence that is homologous to any one of SEQ ID Nos: 1-164. Such homologous sequences may include one or more amino acid substitutions (e.g., 1, 2, 3, 4, 5, 6, 7, or 8 substitutions) and/or share a sequence identify of at least 70%, 75%, 80%, 85%, 90%, or 95% compared to any one of SEQ ID Nos: 1-164. In some aspects, a variant may be capable of mediating secretion of an enzyme when covalently linked to the enzyme and expressed in a Bacillus cell (e.g., in B. subtilis). It is understood that the aforementioned variants may be used in place of SEQ ID NOs: 1-164 in any of the aspects described herein.

In another aspect, the present disclosure relates to an artificially generated SP sequence conjugated in frame with a mature enzyme protein selected from amylase, dehalogenase, lipase, protease, or xylanase, wherein the enzyme protein lacks its nature SP. In other embodiments, the mature enzyme protein is a protein selected from SEQ ID Nos: 165-205, wherein the mature enzyme protein lacks its natural SP.

In yet other aspects, the present disclosure relates to a protein sequence comprising a signal peptide conjugated a mature enzyme, wherein the SP is selected from SEQ ID Nos: 1-164, and the mature enzyme is selected from SEQ ID Nos: 165-205 and is lacking its natural SP.

In still other aspects, present disclosure relates to SPs generated by methods and systems using deep learning. In one embodiment, the SPs are generated by a deep machine learning model that generates functional SPs for protein sequences using a dataset that maps a plurality of known output SP sequences to a plurality of corresponding known input protein sequences. The method may thus, generate, via the trained deep machine learning model, an output SP sequence for an arbitrary input protein sequence. In an exemplary aspect, the trained deep machine learning model is configured to receive the input protein sequence, tokenize each amino acid of the input protein sequence to generate a sequence of token, map the sequence of tokens to a sequence of continuous representations via an encoder, and generate the output SP sequence based on the sequence of continuous representations via a decoder.

In other aspects, the present disclosure relates to a nucleic acid sequence encoding an amino acid sequence selected from SEQ ID Nos: 1-164. In one embodiment, the nucleic acid sequence encodes an amino acid sequence comprising a sequence selected from SEQ ID Nos: 1-164. In yet other embodiments, the nucleic acid sequence encodes a heterologous construct with an amino acid sequence comprising a first sequence selected from SEQ ID Nos: 1-164 and a second sequence selected from SEQ ID Nos: 165-205, wherein the second sequence lacks its natural SP.

In some aspects, the present disclosure relates to a method of expressing a recombinant protein in a host comprising cloning in frame a first nucleotide sequence encoding a signal peptide having an amino acid sequence selected from SEQ ID Nos: 1-164; and a second nucleotide sequence encoding a mature enzyme protein, wherein the mature enzyme protein lacks a natural signal peptide. In an embodiment, the second nucleotide sequence encodes a mature enzyme protein selected from amylase, dehalogenase, lipase, protease, xylanase, or more preferably, the mature enzyme is selected from SEQ ID Nos: 165-205.

It should be noted that the SPs and proteins comprising the SPs are artificial sequences that may be generated through methods and systems using deep learning techniques. These techniques may be implemented in a system comprising a hardware processor. Alternatively, the methods may be implemented using computer executable instructions stored in a non-transitory computer readable medium.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more exemplary aspects of the present disclosure and, together with the detailed description, serve to explain their principles and implementations.

FIG. 1 is a block diagram illustrating a system for generating an SP amino acid sequence using deep learning, in accordance with aspects of the present disclosure.

FIG. 2 illustrates a flow diagram of a method for generating an SP amino acid sequence using deep learning, in accordance with aspects of the present disclosure.

FIG. 3 illustrates an example of a general-purpose computer system on which aspects of the present disclosure can be implemented.

### DETAILED DESCRIPTION

Exemplary aspects are described herein in the context of a system, method, and computer program product for generating a signal peptide (SP) amino acid sequence using deep learning. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the exemplary aspects as illustrated in the accompanying drawings. The same reference indicators will be used to the extent possible throughout the drawings and the following description to refer to the same or like items.

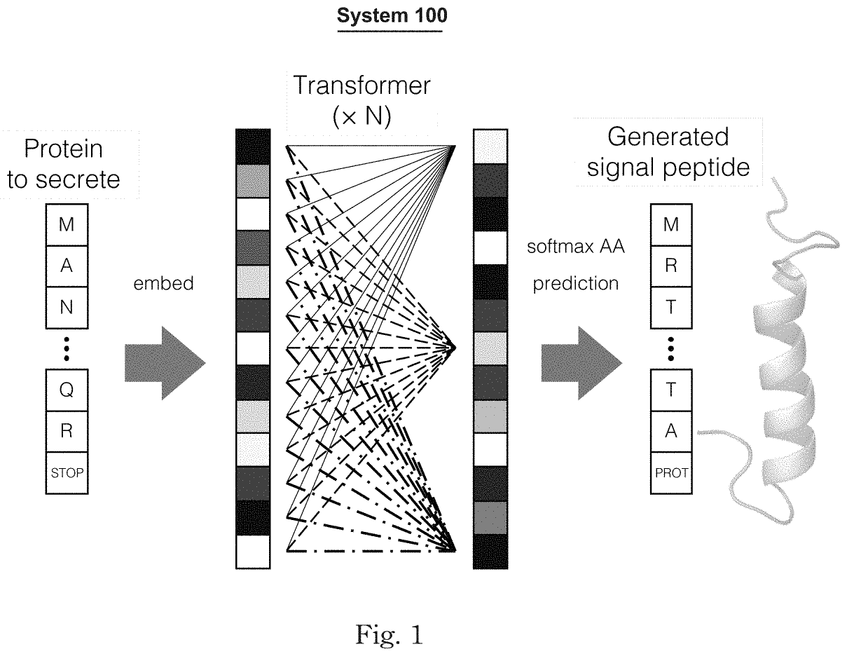

FIG. 1 is a block diagram illustrating system 100 for generating an artificial SP amino acid sequence using deep learning, in accordance with aspects of the present disclosure. System 100 depicts an exemplary deep machine learning model utilized in the present disclosure. In some aspects, the deep machine learning model is an artificial neural network with an encoder-decoder architecture (henceforth, a “transformer”). A transformer is designed to handle ordered sequences of data, such as natural language, for various tasks such as translation. Ultimately, a transformer receives an input sequence and generates an output sequence. Suppose that the input sequence is a sentence. Because a transformer does not require that the input sequence be processed in order, the transformer does not need to process the beginning of a sentence before it processes the end. This allows for parallelization and greater efficiency when compared to counterpart neural networks such as recurrent neural networks. While the present disclosure focuses on transformers having an encoder-decoder architecture, it is understood that in alternative aspects, the methods described herein may instead use an artificial neural network which implements a singular encoder or decoder architecture rather than a paired encoder-decoder architecture. Such architectures may be used to carry out any of the methods described herein.

In some aspects, the dataset used to train the neural network used by the systems described herein may comprise a map which associates a plurality of known output SP sequences to a plurality of corresponding known input protein sequence. For example, the plurality of known input protein sequences used for training may include SEQ ID NO: 206, which is known to have the output SP sequence represented by SEQ ID NO: 207. Another known input protein sequence may be SEQ ID NO: 208, which in turn corresponds to the known output SP sequence represented by SEQ ID NO: 209. SEQ ID NOs: 206-209 are shown in Table 1 below:

TABLE 1
Exemplary known input protein sequences
and known output SP sequences.

| | |
|---|---|
| SEQ ID | AERQPLKIPPIIDVGRGRPVRLDLRPAQTQ |
| NO: 206 | FDKGKLVDVWGVNGQYLAPTVRVKSDDFVK |
| | LTYVNNLPQTVTMNIQGLLAPTDMIGSIHR |
| | KLEAKSSWSPIISIHQPACTCWYHADTMLN |
| | SAFQIYRGLAGMWIIEDEQSKKANLPNKYG |
| | VNDIPLILQDQQLNKQGVQVLDANQKQFFG |
| | KRLFVNGQESAYHQVARGWVRLRIVNASLS |
| | RPYQLRLDNDQPLHLIATGVGMLAEPVPLE |
| | SITLAPSERVEVLVELNEGKTVSLISGQKR |
| | DIFYQAKNLFSDDNELTDNVILELRPEGMA |
| | AVFSNKPSLPPFATEDFQLKIAEERRLIIR |
| | PFDRLINQKRFDPKRIDFNVKQGNVERWYI |
| | TSDEAVGFTLQGAKFLIETRNRQRLPHKQP |
| | AWHDTVWLEKNQEVTLLVRFDHQASAQLPF |
| | TFGVSDFMLRDRGAMGQFIVTE |
| SEQ ID | MMNLTRRQLLTRSAVAATMFSAPKTLWA |
| NO: 207 | |
| SEQ ID | ERIKDLTTIQGVRSNQLIGYGLVVGLDGTG |
| NO: 208 | DQTTQTPFTVQSIVSMMQQMGINLPSGTNL |
| | QLRNVAAVMVTGNLPPFAQPGQPMDVTVSS |
| | MGNARSLRGGTLLMTPLKGADNQVYAMAQG |
| | NLVIGGAGAGASGTSTQINHLGAGRISAGA |
| | IVERAVPSQLTETSTIRLELKEADFSTASM |
| | VVDAINKRFGNGTATPLDGRVIQVQPPMDI |
| | NRIAFIGNLENLDVKPSQGPAKVILNARTG |
| | SVVMNQAVTLDDCAISHGNLSVVINTAPAI |
| | SQPGPFSGGQTVATQVSQVEINKEPGQVIK |
| | LDKGTSLADVVKALNAIGATPQDLVAILQA |
| | MKAAGSLRADLEII |
| SEQ ID | MTLTRPLALISALAALILALPADA |
| NO: 209 | |

Table 1 illustrates two exemplary pairs of known input protein sequences and their respective known output SP sequences. It is understood that the dataset used to train the neural network which generates the artificial SPs described herein may include, e.g., hundreds or thousands of such pairs. A set of known protein sequences, and their respective known SP sequences, can be generated using publicly-accessible databases (e.g., the NCBI or UniProt databases) or proprietary sequencing data. For example, many publicly-accessible databases include annotated polypeptide sequences which identify the start and end position of experimentally validated SPs. In some aspects, the known SP for a given known input protein sequence may be a predicted SP (e.g., identified using a tool such as the SignalP server described in Armenteros, J. et al., “SignalP 5.0 improves signal peptide predictions using deep neural networks.” Nature Biotechnology 37.4 (2019): 420-423.

In some aspects, the neural network used to generate the artificial SPs described herein leverages an attention mechanism, which weighs the relevance of every input (e.g., the amino acid at each position of an input sequence) and draws information from them accordingly when producing the output. The transformer architecture is applied to SP prediction by treating each of the amino acids as a token. The transformer comprises two components: an encoder and decoder. In some aspects, the transformer may comprise a chain of encoders and a chain of decoders. The transformer encoder maps an input sequence of tokens (e.g., the amino acids of an input protein) to a sequence of continuous representations. The sequence of continuous representations is a machine interpretation of the input tokens that relates the positions in each input protein sequence (e.g., of a character) with the positions in each output SP sequence. Given these representations, the decoder then generates an output sequence (comprising the SP amino acids) one token at a time. Each step in this process depends on the generated sequence elements preceding the current step and continues until a special <END OF SP> token is generated. FIG. 1 illustrates this modeling scheme.

In some aspects, the transformer is configured to have multiple layers (e.g., 2-10 layers) and/or hidden dimensions (e.g., 128-2,056 hidden dimensions). For example, the transformer may have 5 layers and a hidden dimension of 550. Each layer may comprise multiple attention heads (e.g., 4-10 attention heads). For example, each layer may comprise 6 attention heads. Training may be performed, for multiple epochs (e.g., 50-200 epochs) with a user-selected dropout rate (e.g., in the range of 0.1-0.8). For example, training may be performed for 100 epochs with a dropout rate of 0.1 in each attention head and after each position-wise feed-forward layer. In some aspects, periodic positional encodings and an optimizer may be used in the transformer. For example, the Adam or Lamb optimizer may be used. In some aspects, the learning rate schedule may include a warmup period followed by exponential or sinusoidal decay. For example, the learning rate can be increased linearly for a first set of batches (e.g., the first 12,500 batches) from 0 to 1e-4 and then decayed by n_steps^{-0.03} after the linear warmup. It should be noted that one skilled in the art may adjust these numerical values to potentially improve the accuracy of functional SP sequence generation.

In some aspects, varying sub-sequences of the input protein sequences may be used as source sequences in order to augment the training dataset, to diminish the effect of choosing one specific length cutoff, and to make the model more robust. For input proteins of length L<105, the model may receive, e.g., the first L−10, L−5, and L residues as training inputs. For mature proteins of L>=105, the model may receive, e.g., the first 95, 100, and 105 amino residues as training inputs. It should be noted that the specific cutoff lengths and amino residues described above may be adjusted for improved accuracy in functional SP sequence generation.

In some aspects, in addition to training on a full dataset, the transformer may be trained on subsets of the full dataset. The subsets may remove sequences with ≥75%, ≥90%, ≥95%, or ≥99% sequence identity to a set of enzymes in order to test the model's ability to generalize to distant protein sequences. Accordingly, the transformer may be trained on a full dataset and truncated versions of a full dataset.

Given a trained deep machine learning model that predicts sequence probabilities, there are various approaches by which PS sequences can be generated. In some aspects, a beam search is applied. A beam search is a heuristic search algorithm that traverses a graph by expanding the most probable node in a limited set. In systems and methods according to the present disclosure, a beam search may be used to generate a sequence by taking the most probable amino acid additions from the N-terminus (i.e., the start of a protein or polypeptide referring to the free amine group located at the end of a polypeptide). In some aspects, a mixed input beam search may be used over the decoder to generate a “generalist” SP, which has the highest probability of functioning across multiple input protein sequences. The beam size for the mixed input beam search may be 5. In traditional implementations of a beam search, the size of the beam refers to the number of unique hypotheses with highest predicted probability for a specific input that are tracked at each generation step. In contrast, the mixed input beam search generates hypotheses for multiple inputs (rather than one), keeping the sequences with highest predicted probabilities.

In some aspects, the trained deep machine learning model may output a SP sequence for an input protein sequence. The output SP sequence may then be queried for novelty (i.e., whether the sequence exists in a database of known functioning SP sequences). In response to determining that the output SP sequence is novel, the output SP sequence may be tested for functionality.

In some aspects, a construct that merges the generated output SP sequence and the input protein sequence is created. The construct is an SP-protein pair whose functionality is evaluated by verifying whether the protein associated with the input protein sequence is localized extracellularly and acquires a native three-dimensional structure that is biologically functional when a signal peptide corresponding to the output SP sequence is present at the amino terminus of the protein. This verification may be performed, e.g., by expressing the SP-protein pair in an industrial gram-positive bacterial host such as Bacillus subtilis, which can be used for secretion of industrial enzymes.

In response to determining that the construct is functional, the SP-protein pair may be deemed functional. In response to determining that the construct is not functional, the deep machine learning model may be further trained to improve the accuracy of SP generation.

As mentioned previously, deep learning has conventionally been used to identify an SP in an enzyme sequences, which comprise SP-protein pairs (e.g., a protein with a corresponding natural SP sequence appended to its amino terminus). The deep machine learning model may be trained using inputs that list the SP-protein pair and indicate the SP in each respective pair. Accordingly, the deep machine learning model learns the characteristics of how SP sequences are positioned relative to the protein sequence and can identify the SP in any arbitrary SP-protein pair. A focus of identification is to determine length and positioning of the SP sequence. In contrast, the generation of SP sequences involves the structure of the SP sequences and the order of characters relative to the characteristics of the protein sequence.

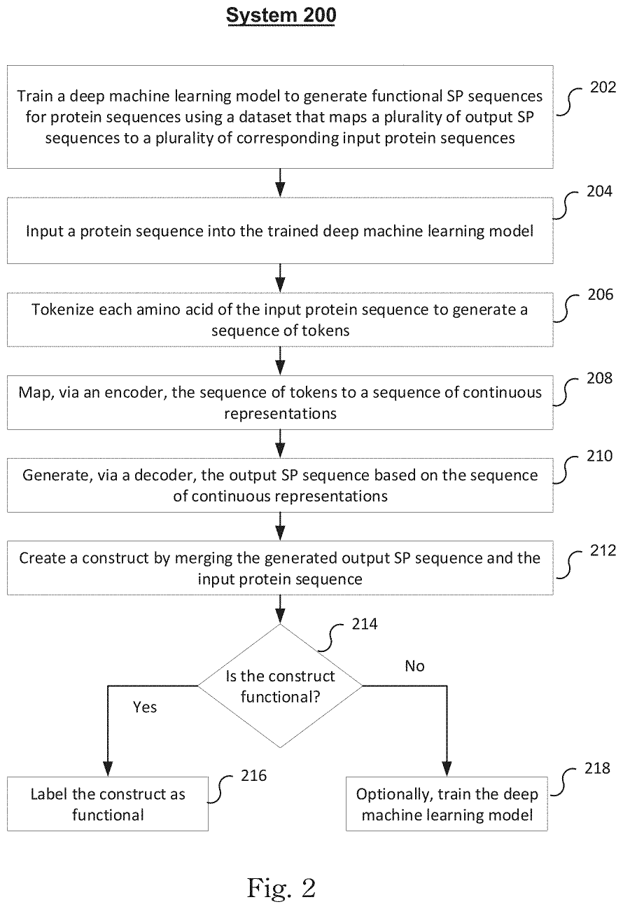

FIG. 2 illustrates a flow diagram of method 200 for generating a SP amino acid sequence using deep learning, in accordance with aspects of the present disclosure. At 202, method 200 trains a deep machine learning model to generate functional SP sequences for protein sequences using a dataset that maps a plurality of output SP sequences to a plurality of corresponding input protein sequences. For example, the deep machine learning model may have a transformer encoder-decoder architecture depicted in system 100.

At 204, method 200 inputs a protein sequence in the trained deep machine learning model. For example, the input protein sequence may be represented by the following sequence:

(SEQ ID NO: 210)
“DGLNGTMMQYYEWHLENDGQHWNRLHDDAAALSDAGITA
IWIPPAYKGNSQADVGYGAYDLYDLGEFNQKGTVRTKYGT
KAQLERAIGSLKSNDINVYGD”.

At 206, the trained deep machine learning model tokenizes each amino acid of the input protein sequence to generate a sequence of tokens. In some aspects, the tokens may be individual characters of the input protein sequence listed above.

At 208, the trained deep machine learning model maps, via an encoder, the sequence of tokens to a sequence of continuous representations. The continuous representations may be machine interpretations of the positions of tokens relative to each other.

At 210, the trained deep machine learning model generates, via a decoder, the output SP sequence based on the sequence of continuous representations. For example, the output SP sequence may be “MKLLTSFVLIGALAFA” (SEQ ID NO: 211).

At 212, method 200 creates a construct by merging the generated output SP sequence and the input protein sequence. The construct in the overarching example may thus be:

(SEQ ID NO: 212)
“MKLLTSFVLIGALAFADGLNGTMMQYYEWHLENDGQH
WNRLHDDAAALSDAGITAIWIPPAYKGNSQADVGYGAY
DLYDLGEFNQKGTVRTKYGTKAQLERAIGSLKSNDINV
YGD”.

At 214, method 200 determines whether the construct is in fact functional. More specifically, method 200 determines whether the protein associated with the input protein sequence “DGLNGTMMQYYEWHLENDGQHWNRLHDDAAALSDAGITAIWIPPAYKGNSQADVG YGAYDLYDLGEFNQKGTVRTKYGTKAQLERAIGSLKSNDINVYGD” (SEQ ID NO: 210) is localized extracellularly and acquires a native three-dimensional structure that is biologically functional when a signal peptide corresponding to the output SP sequence “MKLLTSFVLIGALAFA” (SEQ ID NO: 211) serves as an amino terminus of the protein.

In response to determining that the construct is functional, at 216, method 200 labels the construct as functional. However, in response to determining that the construct is not functional, at 218, method 200 may further train the deep machine learning model. In this particular example, the output SP sequence “MKLLTSFVLIGALAFA” yields a functional construct.

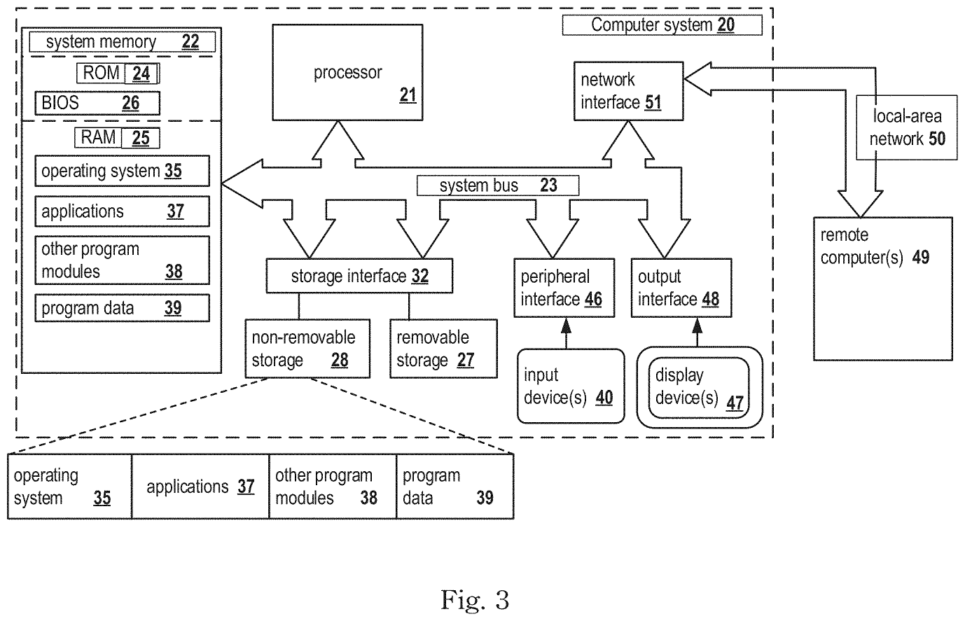

FIG. 3 is a block diagram illustrating a computer system 20 on which aspects of systems and methods for generating a SP amino acid sequence using deep learning may be implemented in accordance with an exemplary aspect. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I²C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-2 may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

Using the above described deep machine learning model, output SPs may be generated which have a high probability of functioning with arbitrary input protein sequences. These input sequences may include, e.g., any protein that is intended to be targeted for a secretion via the Sec or Tat-mediated pathways.

In some embodiments, the protein is an enzyme directed for secretion by the presence of an SP. Such enzymes may include those that are expressed in various microorganisms having industrial applicability in for example, agriculture, chemical synthesis, food production, and pharmaceuticals. These might include, for example, bacteria, fungi, algae, micro algae, yeast, and various eukaryotic hosts (such as Saccharomyces, Pichia, mammalian cells—e.g., CHO or HEK 293 cells). In certain aspects, the microorganism may be a bacteria and may include, but is not limited to, Bacillus, Clostridium, Thermus, Psuedomonas, Acetobacter, Micrococcus, Streptomyces, or a member of the genus Leuconostoc. In a preferred embodiment, the gram-positive bacteria, most preferably Bacillus subtilis.

The enzyme may comprise an enzyme that can be targeted for secretion directed by a SP. In some aspects, the enzyme is an amylase, dehalogenase, lipase, protease, or xylanase. In some embodiments, the input sequence used to generate an SP comprises a sequence of an enzyme found in Table 2 (e.g., any one of SEQ ID NOs: 165-205):

TABLE 2
Input Sequences

| Enzyme | Accession No. | SEQ ID NO. |
|---|---|---|
| Amylase | AAA22240.1 | 165 |
| | BAB71820.1 | 166 |
| | ABL75259.1 | 167 |
| | ABW34932.1 | 168 |
| | AFI62032.1 | 169 |
| | A0A1C7DFY9 | 170 |
| | A0A1C7DR68 | 171 |
| | R8CT19 | 172 |
| | A0A0K9GFU6 | 173 |
| | A0A143H9P3 | 174 |
| | A9UJ60 | 175 |
| | C6GKH6 | 176 |
| | A0A0Q4KKJ4 | 177 |
| | A0A0Q6I034 | 178 |
| | O82839 | 179 |
| Dehalogenase | ACJ24902.1 | 180 |
| | AIQ78389.1 | 181 |
| | OBG15055.1 | 182 |
| | A0A1Z3HGC4 | 183 |
| | B8H3S9 | 184 |
| | Q9ZER0 | 185 |
| Lipase | AAB01071.1 | 186 |
| | ABC48693.1 | 187 |
| | P37957 | 188 |
| | Q79F14 | 189 |
| | O59952 | 190 |
| | U3AVP1 | 191 |
| | F8H9H4 | 192 |
| | F6LQK7 | 193 |
| | H0TLU8 | 194 |
| | I0RVG3 | 195 |
| Protease | AGS78407.1 | 196 |
| | P04189 | 197 |
| | P00782 | 198 |
| | G9JKM6 | 199 |
| | P27693 | 200 |
| Xylanase | ANC94865.1 | 201 |
| | Q9P8J1 | 202 |
| | P00694 | 203 |
| | A0A0S1S264 | 204 |
| | W8VR85 | 205 |

The sequences provided in Table 2 above do not include the naturally-occurring SP associated with each of these enzyme. In the present application, the input sequence are presented into the machine deep learning system without its natural SP. Those of skill in the art would understand, based on the information provided for each of the known enzymes, that the SPs are removed following secretion and they would be capable of discerning the sequences based on the information provided in each of the protein databases. Thus, in one embodiment, the output SPs generated will be conjugated to an amylase, dehalogenase, lipase, protease, or xylanase enzyme lacking its corresponding natural SP.

Upon training of the neural networks, the output SP sequences generated may include an amino acid sequence having an amino acid length in the range of 4-70 amino acids. Like classic SPs, the output sequences may have a N-region with positively charged residues, a H-region having alpha-helix forming residues, and a C-region having polar or non-charged residues. In some embodiments, the output SP sequence may be selected from the sequences listed on the following Table 3:

TABLE 3
Output SP Sequences

| SP | SEQ | |
|---|---|---|
| Name | ID | SP Amino Acid Sequence |
| sps1-1 | 1 | MRFFGIHLALALATTSFA |
| sps1-2 | 2 | MRQLFTSLLALLGVCSLA |
| sps1-3 | 3 | MKLSLKSIILLPTVAT |
| sps1-4 | 4 | MKKPLGKIVASTALLISVAFSSSIASA |
| sps2-1 | 5 | MVATPFYLFLPWGVVAALVRSQA |
| sps2-2 | 6 | MKFFNPFKVIALACISGALATAQA |
| sps2-3 | 7 | MKKVLLATAAATLSGLMAAHA |
| sps2-4 | 8 | MIKKIPLKTIAVMALSGCTFFVNG |
| sps3-1 | 9 | MRLIVFLATSATSLFASLA |
| sps3-2 | 10 | MFKLKDILIGLTGILLSSLFA |
| sps3-3 | 11 | MLHVALLLIIGTTCSSIVSA |
| sps3-4 | 12 | MRLAKIAGLTASLLFSLWGALA |
| sps4-1 | 13 | MVGYSTAWLLLLAASVIASG |
| sps4-2 | 14 | MAVNTKLIGVSLYSFTPFLVFA |
| sps4-3 | 15 | MLGRGALTAAILAGVATADS |
| sps4-4 | 16 | MAILVLLFLLAVEINS |
| sps5-1 | 17 | MLLPAFMLLILPAALA |
| sps5-2 | 18 | MKMRTGKKGFLSILLAFLLV |
| | | ITSIPFTLVDVEA |
| sps5-3 | 19 | MSNKPAKCLAVLAAIATLSATQA |
| sps5-4 | 20 | MKMRTGKKGFLSILLAFLLVIT |
| | | SIPFTLVDVEA |
| sps6-1 | 21 | MKLGFLTSFVAYLTSAA |
| sps6-2 | 22 | MKLSTIFVRFLAIALLATMSTAQA |
| sps6-3 | 23 | MQRSLFLVLSLVSSVASA |
| sps6-4 | 24 | MKLFTATIAVLGAVSATAHA |
| sps7-1 | 25 | MLKNFLLASLAICVTFSATG |
| sps7-2 | 26 | MVKNFQKILVLALLIVCCSSISLATFA |
| sps7-3 | 27 | MKLLPAFFLITAATVASA |
| sps7-4 | 28 | MKDLFRLIALLSCCLALFPLTFA |
| sps8-1 | 29 | MRKTAVSFTVCALMLGTAMA |
| sps8-2 | 30 | MKKFCKILVISMLAVLGLTPAAVA |
| sps8-3 | 31 | MKKSLSAVLLGVALSAVASSAFA |
| sps8-4 | 32 | MKSLLLTAFAAGTALA |
| sps9-1 | 33 | MLSLKSLFLSTLLIVLAASGFA |
| sps9-2 | 34 | MKKRLHIGLLLSLIAFQAGFA |
| sps9-3 | 35 | MKLLAFIFALFLFSIARA |
| sps9-4 | 36 | MNKLFYLFMLGLAAFA |
| sps10-1 | 37 | MKFSTILAAAILVGVRA |
| sps10-2 | 38 | MKVFTLAFAIICQLFASA |
| sps10-3 | 39 | MKKKIAIILMSLLLNTIASTFA |
| sps10-4 | 40 | MKLKIVFAVAAIAPVLHS |
| sps11-1 | 41 | MVYTSILLAASAATVQA |
| sps11-2 | 42 | MNKTIVLAASLLGLFSSTALA |
| sps11-3 | 43 | MLKLILALCFSLPFAALA |
| sps11-4 | 44 | MKFTQAVLSLLGSAATALA |
| sps12-1 | 45 | MGFRLKALLVGCLIFLAVSSAIA |
| sps12-2 | 46 | MTSYEFLLVILGVLLSGA |
| sps12-3 | 47 | MPMTLLVLSLLATLFGSWVA |
| sps12-4 | 48 | MNIRLGALLAGLLLSAMASAVFA |
| sps13-1 | 49 | MKNLLFSTLTAVLITSVSFA |
| sps13-2 | 50 | MKKFAVICGLLFACIVDA |
| sps13-3 | 51 | MNKKFKTIMALAIATLSAAGVGVAHA |
| sps13-4 | 52 | MKKSLISFLALGLLFGSAFA |
| sps14-1 | 53 | MALANKFFLLVALGLSVSG |
| sps14-2 | 54 | MVIVLTSIILALWNAQA |
| sps14-3 | 55 | MTKFLLSLAVLATAVASA |
| sps14-4 | 56 | MKFLSIVLLIVGLAYG |
| sps15-1 | 57 | MMAAVVRAVAATLILILCGAELA |
| sps15-2 | 58 | MLPTAAFLSVNLLLTGAFFGCA |
| sps15-3 | 59 | MYSLIPSLAVLAALSFAVSA |
| sps15-4 | 60 | MFKFVLVLSVLAALASARA |
| sps16-1 | 61 | MRVPYLIASLLALAVSLFSTATA |
| sps16-2 | 62 | MKKIKSILVLALIGIMSSALA |
| sps16-3 | 63 | MLGAKFLWTVLFSLSLSLAHA |
| sps16-4 | 64 | MLTFHRIIRKGWMFLLAFLLTA |
| | | LLFCPTGQPAKA |
| sps17-1 | 65 | MLIRKYLSFAISLLIATALPASA |
| sps17-2 | 66 | MEKVLLRLLILLSLLAGALSFA |
| sps17-3 | 67 | MKLGSIFLFALFLACSAEA |
| sps17-4 | 68 | MNLKILFALALGVCLAA |
| sps18-1 | 69 | MTRPAPAFRLSLVILCLAIPAADA |
| sps18-2 | 70 | MVTMKLRLIALAVCLCTFINASFA |
| sps18-3 | 71 | MTKLLAVIAASLMFAASTFA |
| sps18-4 | 72 | MVSNKRVLALSALFGCCSLASA |
| sps19-1 | 73 | MVSFKSALFAAAAVATVADA |
| sps19-2 | 74 | MQKKTAIAIAAGTAIATVAAGTQA |
| sps19-3 | 75 | MVSFSSLLAAASLAVVNA |
| sps19-4 | 76 | MKNFATLSAVLAGATALA |
| sps20-1 | 77 | MKLNKLLSIAAGCTVLGSTYALA |
| sps20-2 | 78 | MKLKKLGVILAICLGISSTFA |
| sps20-3 | 79 | MKKLLLAACVLFSLASVSA |
| sps20-4 | 80 | MIRLKRLLAGLLLPLFVTAFG |
| sps21-1 | 81 | MTRSLFIFSLLALAIFSGVSASA |
| sps21-2 | 82 | MKLIPNKKTLIAGILAISTSFAYS |
| sps21-3 | 83 | MLKRFVKLAVIALAFAYVSA |
| sps21-4 | 84 | MKKTGFIGKTLALVIAAGMAGTAAFA |
| sps22-1 | 85 | MKLGKLLASVAATLGVSGVNA |
| sps22-2 | 86 | MKKLLILACLLISSLES |
| sps22-3 | 87 | MTKFLLSLIFITIASALA |
| sps22-4 | 88 | MKKTILALALLGSLAA |
| sps23-1 | 89 | MRSLGFTFLISALFGVSLSA |
| sps23-2 | 90 | MKPACRLISLLMLAVSGIASA |
| sps23-3 | 91 | MMLTFFISLLFLSSALA |
| sps23-4 | 92 | MTLKTTITLFFAALSANAAFA |
| sps24-1 | 93 | MRAKALAASLAGALAGAASA |
| sps24-2 | 94 | MVSLSFSLVASAVTHVASA |
| sps24-3 | 95 | MVSFSSLNALFLATVLA |
| sps24-4 | 96 | MKFQDLTLVLSLSTALA |
| sps25-1 | 97 | MRVLSATAFLALLAFGLSGATA |
| sps25-2 | 98 | MKFLSTAFVLLIALVAGCSTA |
| sps25-3 | 99 | MLKRFLTLFLGFLALASSLA |
| sps25-4 | 100 | MKLLTSFVLIGALAFA |
| sps26-1 | 101 | MLKKLAMAVGAMLTSISFLLPSSAQA |
| sps26-2 | 102 | MKKLLVIAALACGVATAQA |
| sps26-3 | 103 | MIKTLLVSSILIPCLATGA |
| sps26-4 | 104 | MGIQKKVSILVAGLFMATAFATA |
| sps27-1 | 105 | MKKIVALFLVFCFLAG |
| sps27-2 | 106 | MNKKVLAAIVLGMLSVFTSAAQA |
| sps27-3 | 107 | MKKTAIASALLALPFVFA |
| sps27-4 | 108 | MKKTAAIAALAGLSFAGMAHA |
| sps28-1 | 109 | MISANKILFLILCVACVSA |
| sps28-2 | 110 | MVKLASILLIILAGESFA |
| sps28-3 | 111 | MINKLIALTVLFSLGINA |
| sps28-4 | 112 | MVASLWSSILPVLAFLWADLSAGA |
| sps29-1 | 113 | MKFLLFIALSLAVATAA |
| sps29-2 | 114 | MRHFLSLLLYGATLVSSSACS |
| sps29-3 | 115 | MKFSAIVLLAALAFAVSA |
| sps29-4 | 116 | MKKRLLIASVALGSLFSFCA |
| sps30-1 | 117 | MSWRSIFLLVLLASIDFING |
| sps30-2 | 118 | MRLPSLLLPLAALIA |
| sps30-3 | 119 | MKVLAALVLALVATASA |
| sps30-4 | 120 | MARA |
| sps31-1 | 121 | MRKLLIWLAGFLVLILKT |
| sps31-2 | 122 | MRKFISSLLLGLVVSIATAVA |
| sps31-3 | 123 | MNTLFLFTSLFLFLFAKVTA |
| sps31-4 | 124 | MKFLILLITLGAIAATALA |
| sps32-1 | 125 | MRVTSKVILTLIAATAFATAFTWSA |
| sps32-2 | 126 | MKKFKRTILSGLALAMSIAQA |
| sps32-3 | 127 | MLFKSVLLALASAGVAVNA |
| sps32-4 | 128 | MKLFKILTACLFIGLLNVSA |
| sps33-1 | 129 | MAVMRFFASLPRRVA |
| sps33-2 | 130 | MLKRAAFLVGVSLAVAAGCGPAQA |
| sps33-3 | 131 | MTHRTFAALPAAALAAVSSAAFA |
| sps33-4 | 132 | MKLSQSLTYLAVLGLAAGANA |
| sps34-1 | 133 | MASKLAFFLALALAAAA |
| sps34-2 | 134 | MKFLSLKLVVLAFYVAFQINA |
| sps34-3 | 135 | MAKLIALVLLGLAAA |
| sps34-4 | 136 | MRSLLLTLLGALLRA |
| sps35-1 | 137 | MKLNIVKLLVLAAFAQAASA |
| sps35-2 | 138 | MILFYVLPVVLALVSG |
| sps35-3 | 139 | MKKNLLKLTLALISGMSQFA |
| sps35-4 | 140 | MKFLIPLFVLFIVFGNAYA |
| sps36-1 | 141 | MKRVFSLFTAVCGLLSVSA |
| sps36-2 | 142 | MKKFSIFLVLSITVLA |
| sps36-3 | 143 | MKKKIVAVLTLSVVLA |
| sps36-4 | 144 | MKKRVISALAALWLSVLGAPAVLA |
| sps37-1 | 145 | MGVFSFLTTEAMAVFLAGLAHA |
| sps37-2 | 146 | MTMKGLRVVALVVLASLGIFA |
| sps37-3 | 147 | MTKFLSASLALLSGLATASSDA |
| sps37-4 | 148 | MTQKSLLLALTAVALVSVNA |
| sps38-1 | 149 | MNRLYAVFAVLCFAQVLHG |
| sps38-2 | 150 | MKKLLLQSLILSELGGCLA |
| sps38-3 | 151 | MAARSVLLLALLTLAVSTA |
| sps38-4 | 152 | MKGTLAFLLVFLLNLYVHG |
| sps39-1 | 153 | MLSIDTSSTRRVVPNTALFPNTHRR |
| | | DFATAGQLLAMASAVLTGAPAHA |
| sps39-2 | 154 | MNISIFVGKLALAALGSALVA |
| sps39-3 | 155 | MRRLFLLSSLASLSVASA |
| sps39-4 | 156 | MKCCRIMFVLLGLWFVFGLSVPGGRTEA |
| sps40-1 | 157 | MKFLILATLSIFTGILA |
| sps40-2 | 158 | MKVFTLAFFLAIIVSQA |
| sps40-3 | 159 | MKKKIAITLLFLSLLNRA |
| sps40-4 | 160 | MKLLKVIATAFLGLTSFASA |
| sps41-1 | 161 | MPTVVALDLATYVLQPSKRA |
| sps41-2 | 162 | MLMVPLLLALGAVAAG |
| sps41-3 | 163 | MPAARRLALFAAVALAAVGLSPAALA |
| sps41-4 | 164 | MRSLLLTSALAALVSLAAASA |

### EXAMPLES

Bacterial Strains, DNA Design, and Library Construction

An expression vector was constructed from the Bacillus subtilis shuttle vector pHT01 by removal of the BsaI restriction sites and replacing the inducible Pgrac promotor with the constitutive promotor Pveg. However, IPTG was included during expression to ensure no residual or off-site inhibition from the Lad fragment still included on the pHT vector. SP sequences predicted from the machine deep learning model were reverse translated into DNA sequences for synthesis using JCat39 for codon optimization with Bacillus subtilis (strain 168). Each gene of interest was modeled at four homology cutoffs resulting in 4 predicted signal peptides. These 4 signal peptides were synthesized as a single DNA fragment with spacers including the BsaI restriction sites. 8 individual colonies were picked from each group of 4 predicted SPs. Protein sequences were selected from literature reports of enzymes expressed in Bacillus host systems. Table 1 lists the enzymes used. Signal peptide and protein DNA sequences were ordered from Twist Biosciences and cloned into their E. coli cloning vector. Bacillus subtilis PY97 was the base strain used for the expression of enzymes. Native enzymes that could interfere with measurement were knocked.

The expression vector backbone, gene of interest, and SP fragments were amplified via PCR with primers including BsaI sites and assembled with a linker GGGGCT sequence (encoding Glycine and Alanine) between the generated SP and the target protein. Each linear DNA fragment was agarose gel purified. The reactions were performed with 700 ng vector PCR product, 100 ng signal peptide group PCR product, and 300 ng gene of interest PCR product in 20 μl reactions (2 μl 10× T4 Ligase Buffer, 2 μl 10×BSA, 0.8 μl BsaI-HFv2, 1 μl T4 Ligase). The reactions were cycled 35 times (10 min, 37° C.; 5 min, 16° C.) then heat inactivated (5 min, 50° C.; 5 min, 80° C.) before being stored at 4° C. for use directly.

Enzyme Expression and Functional Characterization.

All Bacillus strains were transformed by natural competency as previously described. Transformations were plated on LB agar (10 g/l tryptone, 5 g/l yeast extract, 10 g/l NaCl, 15 g/l agar) supplemented with 5 μg/ml chloramphenicol and grown overnight at 37° C. Single colonies were picked and grown overnight in 96-well plates with LB containing 17 μg/ml chloramphenicol then stored as glycerol stocks. For enzyme expression, cultures were seeded from glycerol stocks into 100 μl LB media and grown overnight at 37° C. A 10 μl aliquot of the overnight culture was trans-ferred into 500 μl of 2×YT media (16 g/l Tryptone, 10 g/l yeast extract, 5 g/l NaCl) containing 1 mM IPTG and incu-bated for 48 hrs at either 30° C. or 37° C. with shaking (900 rpm, 3 mm throw). Culture supernatants were clarified by centrifugation (4000 rpm, 10 min) and used directly in enzyme activity assays. Strains were grown and expressed in at least three biological replicates from each original picked colony.

Enzyme expression quantification was attempted via SDS-PAGE but the observed expression level was below a quantifiable limit. Enzyme expression was too low to reliably quantify with SDS-PAGE, so the relative expression of each enzyme was approximated by activity measurements. Enzyme activity was measured in the linear response range for each substrate and reaction condition. Intracellular enzyme expression was assessed by washing the cell pellet after the supernatant was removed, and then resuspending in 500 μl of 50 mM HEPES buffer with 2 mg/ml Lysozyme and incubated for 30 minutes at 37° C. The resuspended material was centrifuged again and used directly in enzyme activity assays.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

1. A peptide sequence comprising an amino acid sequence selected from any one of SEQ ID Nos:1-164, wherein the peptide is a signal peptide.

2. The peptide sequence according to claim 1, wherein the signal peptide is capable of mediating secretion of an enzyme when covalently linked to the enzyme and expressed in a gram-positive bacterial host cell.

3. The peptide sequence according to claim 1, wherein the signal peptide is capable of mediating secretion of an enzyme when covalently linked to the enzyme and expressed in a Bacillus cell.

4. The peptide sequence according to claim 1, wherein the signal peptide is capable of mediating secretion of an enzyme when covalently linked to the enzyme and expressed in a Bacillus subtilis cell.

5. The peptide sequence according to claim 1, wherein the signal peptide is capable of mediating secretion of a functional enzyme when covalently linked to the enzyme and expressed in a Bacillus cell.

6. The peptide sequence according to claim 2, wherein the enzyme is selected from an amylase, a dehalogenase, a lipase, a protease, or a xylanase.

7. The peptide sequence according to claim 2, wherein the enzyme is an amylase selected from any one of SEQ ID Nos: 165-179.

8. The peptide sequence according to claim 2, wherein the enzyme is a dehalogenase selected from any one of SEQ ID Nos: 180-185.

9. The peptide sequence according to claim 2, wherein the enzyme is a lipase selected from any one of SEQ ID Nos: 186-195.

10. The peptide sequence according to claim 2, wherein the enzyme is a protease selected from any one of SEQ ID Nos: 196-200.

11. The peptide sequence according to claim 2, wherein the enzyme is a xylanase selected from any one of SEQ ID Nos: 201-205.

12. A peptide sequence comprising an amino acid sequence selected from any one of SEQ ID Nos:1-164.

13. A peptide sequence comprising an amino acid sequence that is a variant of any one of SEQ ID Nos:1-164, wherein the variant comprises:
a) a truncated subsequence present in any one of SEQ ID Nos:1-164, and/or
b) a sequence homologous to any one of SEQ ID Nos:1-164;
wherein the variant is capable of mediating secretion of an enzyme when covalently linked to the enzyme and expressed in a Bacillus cell.

14. A protein sequence comprising a signal peptide conjugated to a mature enzyme, wherein the signal peptide is selected from any one of SEQ ID Nos: 1-164, and the mature enzyme is selected from any one of SEQ ID Nos: 165-205.

15. A signal peptide (SP) comprising an amino acid sequence selected from any one of SEQ ID Nos: 1-164, generated by a deep machine learning method, wherein the method comprises:
training a deep machine learning model to generate functional SP sequences for protein sequences using a dataset that maps a plurality of input protein sequence to a plurality of corresponding output SP sequences;
generating, via the trained deep machine learning model, an output SP sequence for an input protein sequence, wherein the trained deep machine learning model is configured to:
receive the input protein sequence;
tokenize each amino acid of the input protein sequence to generate a sequence of tokens;
map, via an encoder, the sequence of tokens to a sequence of continuous representations; and
generate, via a decoder, the output SP sequence based on the sequence of continuous representations.

16. A nucleic acid sequence encoding the amino acid sequence of any one of SEQ ID No: 1-164.

17. A method of expressing a recombinant protein in a host cell, the method comprising:
cloning in frame a first nucleotide sequence encoding an SP having an amino acid sequence selected from any one of SEQ ID Nos: 1-164, and a second nucleotide sequence encoding a mature enzyme protein, wherein the mature enzyme protein lacks a natural SP; and
expressing the recombinant protein in the host cell, wherein the recombinant protein comprises the SP having an amino acid sequence selected from any one of SEQ ID Nos: 1-164, covalently linked to the mature enzyme protein.

18. The method according to claim 17, wherein the second nucleotide sequence encodes a mature enzyme protein selected from an amylase, a dehalogenase, a lipase, a protease, or a xylanase.

19. The method according to claim 17, wherein the second nucleotide sequence encodes a mature enzyme protein having an amino acid sequence selected from SEQ ID Nos: 165-205.

20. The method according to claim 17, wherein the host cell is a gram-positive bacteria.

21. The method according to claim 17, wherein the host cell is a member of the Bacillus genus.

22. The method according to claim 17, wherein the host cell is a Bacillus subtilis cell.


SEQUENCE LISTING (212 sequences)

SEQ ID NO: 1 - protein, 18 aa, Artificial Sequence
  N-terminal Signal Peptide
MRFFGIHLALALATTSFA

SEQ ID NO: 2 - protein, 18 aa, Artificial Sequence
  N-terminal Signal Peptide
MRQLFTSLLALLGVCSLA

SEQ ID NO: 3 - protein, 16 aa, Artificial Sequence
  N-terminal Signal Peptide
MKLSLKSIILLPTVAT

SEQ ID NO: 4 - protein, 27 aa, Artificial Sequence
  N-terminal Signal Peptide
MKKPLGKIVASTALLISVAFSSSIASA

SEQ ID NO: 5 - protein, 23 aa, Artificial Sequence
  N-terminal Signal Peptide
MVATPFYLFLPWGVVAALVRSQA

SEQ ID NO: 6 - protein, 24 aa, Artificial Sequence
  N-terminal Signal Peptide
MKFFNPFKVIALACISGALATAQA

SEQ ID NO: 7 - protein, 21 aa, Artificial Sequence
  N-terminal Signal Peptide
MKKVLLATAAATLSGLMAAHA

SEQ ID NO: 8 - protein, 24 aa, Artificial Sequence
  N-terminal Signal Peptide
MIKKIPLKTIAVMALSGCTFFVNG

SEQ ID NO: 9 - protein, 19 aa, Artificial Sequence
  N-terminal Signal Peptide
MRLIVFLATSATSLFASLA

SEQ ID NO: 10 - protein, 21 aa, Artificial Sequence
  N-terminal Signal Peptide
MFKLKDILIGLTGILLSSLFA

SEQ ID NO: 11 - protein, 20 aa, Artificial Sequence
  N-terminal Signal Peptide
MLHVALLLIIGTTCSSIVSA

SEQ ID NO: 12 - protein, 22 aa, Artificial Sequence
  N-terminal Signal Peptide
MRLAKIAGLTASLLFSLWGALA

SEQ ID NO: 13 - protein, 20 aa, Artificial Sequence
  N-terminal Signal Peptide
MVGYSTAWLLLLAASVIASG

SEQ ID NO: 14 - protein, 22 aa, Artificial Sequence
  N-terminal Signal Peptide
MAVNTKLIGVSLYSFTPFLVFA

SEQ ID NO: 15 - protein, 20 aa, Artificial Sequence
  N-terminal Signal Peptide
MLGRGALTAAILAGVATADS

SEQ ID NO: 16 - protein, 16 aa, Artificial Sequence
  N-terminal Signal Peptide
MAILVLLFLLAVEINS

SEQ ID NO: 17 - protein, 16 aa, Artificial Sequence
  N-terminal Signal Peptide
MLLPAFMLLILPAALA

SEQ ID NO: 18 - protein, 33 aa, Artificial Sequence
  N-terminal Signal Peptide
MKMRTGKKGFLSILLAFLLVITSIPFTLVDVEA

SEQ ID NO: 19 - protein, 23 aa, Artificial Sequence
  N-terminal Signal Peptide
MSNKPAKCLAVLAAIATLSATQA

SEQ ID NO: 20 - protein, 33 aa, Artificial Sequence; identical to SEQ ID NO: 18
  N-terminal Signal Peptide

SEQ ID NO: 21 - protein, 17 aa, Artificial Sequence
  N-terminal Signal Peptide
MKLGFLTSFVAYLTSAA

SEQ ID NO: 22 - protein, 24 aa, Artificial Sequence
  N-terminal Signal Peptide
MKLSTIFVRFLAIALLATMSTAQA

SEQ ID NO: 23 - protein, 18 aa, Artificial Sequence
  N-terminal Signal Peptide
MQRSLFLVLSLVSSVASA

SEQ ID NO: 24 - protein, 20 aa, Artificial Sequence
  N-terminal Signal Peptide
MKLFTATIAVLGAVSATAHA

SEQ ID NO: 25 - protein, 20 aa, Artificial Sequence
  N-terminal Signal Peptide
MLKNFLLASLAICVTFSATG

SEQ ID NO: 26 - protein, 27 aa, Artificial Sequence
  N-terminal Signal Peptide
MVKNFQKILVLALLIVCCSSISLATFA

SEQ ID NO: 27 - protein, 18 aa, Artificial Sequence
  N-terminal Signal Peptide
MKLLPAFFLITAATVASA

SEQ ID NO: 28 - protein, 23 aa, Artificial Sequence
  N-terminal Signal Peptide
MKDLFRLIALLSCCLALFPLTFA

SEQ ID NO: 29 - protein, 20 aa, Artificial Sequence
  N-terminal Signal Peptide
MRKTAVSFTVCALMLGTAMA

SEQ ID NO: 30 - protein, 24 aa, Artificial Sequence
  N-terminal Signal Peptide
MKKFCKILVISMLAVLGLTPAAVA

SEQ ID NO: 31 - protein, 23 aa, Artificial Sequence
  N-terminal Signal Peptide
MKKSLSAVLLGVALSAVASSAFA

SEQ ID NO: 32 - protein, 16 aa, Artificial Sequence
  N-terminal Signal Peptide
MKSLLLTAFAAGTALA

SEQ ID NO: 33 - protein, 22 aa, Artificial Sequence
  N-terminal Signal Peptide
MLSLKSLFLSTLLIVLAASGFA

SEQ ID NO: 34 - protein, 21 aa, Artificial Sequence
  N-terminal Signal Peptide
MKKRLHIGLLLSLIAFQAGFA

SEQ ID NO: 35 - protein, 18 aa, Artificial Sequence
  N-terminal Signal Peptide
MKLLAFIFALFLFSIARA

SEQ ID NO: 36 - protein, 16 aa, Artificial Sequence
  N-terminal Signal Peptide
MNKLFYLFMLGLAAFA

SEQ ID NO: 37 - protein, 17 aa, Artificial Sequence
  N-terminal Signal Peptide
MKFSTILAAAILVGVRA

SEQ ID NO: 38 - protein, 18 aa, Artificial Sequence
  N-terminal Signal Peptide
MKVFTLAFAIICQLFASA

SEQ ID NO: 39 - protein, 22 aa, Artificial Sequence
  N-terminal Signal Peptide
MKKKIAIILMSLLLNTIASTFA

SEQ ID NO: 40 - protein, 18 aa, Artificial Sequence
  N-terminal Signal Peptide
MKLKIVFAVAAIAPVLHS

SEQ ID NO: 41 - protein, 17 aa, Artificial Sequence
  N-terminal Signal Peptide
MVYTSILLAASAATVQA

SEQ ID NO: 42 - protein, 21 aa, Artificial Sequence
  N-terminal Signal Peptide
MNKTIVLAASLLGLFSSTALA

SEQ ID NO: 43 - protein, 18 aa, Artificial Sequence
  N-terminal Signal Peptide
MLKLILALCFSLPFAALA

SEQ ID NO: 44 - protein, 19 aa, Artificial Sequence
  N-terminal Signal Peptide
MKFTQAVLSLLGSAATALA

SEQ ID NO: 45 - protein, 23 aa, Artificial Sequence
  N-terminal Signal Peptide
MGFRLKALLVGCLIFLAVSSAIA

SEQ ID NO: 46 - protein, 18 aa, Artificial Sequence
  N-terminal Signal Peptide
MTSYEFLLVILGVLLSGA

SEQ ID NO: 47 - protein, 20 aa, Artificial Sequence
  N-terminal Signal Peptide
MPMTLLVLSLLATLFGSWVA

SEQ ID NO: 48 - protein, 23 aa, Artificial Sequence
  N-terminal Signal Peptide
MNIRLGALLAGLLLSAMASAVFA

SEQ ID NO: 49 - protein, 20 aa, Artificial Sequence
  N-terminal Signal Peptide
MKNLLFSTLTAVLITSVSFA

SEQ ID NO: 50 - protein, 18 aa, Artificial Sequence
  N-terminal Signal Peptide
MKKFAVICGLLFACIVDA

SEQ ID NO: 51 - protein, 26 aa, Artificial Sequence
  N-terminal Signal Peptide
MNKKFKTIMALAIATLSAAGVGVAHA

SEQ ID NO: 52 - protein, 20 aa, Artificial Sequence
  N-terminal Signal Peptide
MKKSLISFLALGLLFGSAFA

SEQ ID NO: 53 - protein, 19 aa, Artificial Sequence
  N-terminal Signal Peptide
MALANKFFLLVALGLSVSG

SEQ ID NO: 54 - protein, 17 aa, Artificial Sequence
  N-terminal Signal Peptide
MVIVLTSIILALWNAQA

SEQ ID NO: 55 - protein, 18 aa, Artificial Sequence
  N-terminal Signal Peptide
MTKFLLSLAVLATAVASA

SEQ ID NO: 56 - protein, 16 aa, Artificial Sequence
  N-terminal Signal Peptide
MKFLSIVLLIVGLAYG

SEQ ID NO: 57 - protein, 23 aa, Artificial Sequence
  N-terminal Signal Peptide
MMAAVVRAVAATLILILCGAELA

SEQ ID NO: 58 - protein, 22 aa, Artificial Sequence
  N-terminal Signal Peptide
MLPTAAFLSVNLLLTGAFFGCA

SEQ ID NO: 59 - protein, 20 aa, Artificial Sequence
  N-terminal Signal Peptide
MYSLIPSLAVLAALSFAVSA

SEQ ID NO: 60 - protein, 19 aa, Artificial Sequence
  N-terminal Signal Peptide
MFKFVLVLSVLAALASARA

SEQ ID NO: 61 - protein, 23 aa, Artificial Sequence
  N-terminal Signal Peptide
MRVPYLIASLLALAVSLFSTATA

SEQ ID NO: 62 - protein, 21 aa, Artificial Sequence
  N-terminal Signal Peptide
MKKIKSILVLALIGIMSSALA

SEQ ID NO: 63 - protein, 21 aa, Artificial Sequence
  N-terminal Signal Peptide
MLGAKFLWTVLFSLSLSLAHA

SEQ ID NO: 64 - protein, 34 aa, Artificial Sequence
  N-terminal Signal Peptide
MLTFHRIIRKGWMFLLAFLLTALLFCPTGQPAKA

SEQ ID NO: 65 - protein, 23 aa, Artificial Sequence
  N-terminal Signal Peptide
MLIRKYLSFAISLLIATALPASA

SEQ ID NO: 66 - protein, 22 aa, Artificial Sequence
  N-terminal Signal Peptide
MEKVLLRLLILLSLLAGALSFA

SEQ ID NO: 67 - protein, 19 aa, Artificial Sequence
  N-terminal Signal Peptide
MKLGSIFLFALFLACSAEA

SEQ ID NO: 68 - protein, 17 aa, Artificial Sequence
  N-terminal Signal Peptide
MNLKILFALALGVCLAA

SEQ ID NO: 69 - protein, 24 aa, Artificial Sequence
  N-terminal Signal Peptide
MTRPAPAFRLSLVILCLAIPAADA

SEQ ID NO: 70 - protein, 24 aa, Artificial Sequence
  N-terminal Signal Peptide
MVTMKLRLIALAVCLCTFINASFA

SEQ ID NO: 71 - protein, 20 aa, Artificial Sequence
  N-terminal Signal Peptide
MTKLLAVIAASLMFAASTFA

SEQ ID NO: 72 - protein, 22 aa, Artificial Sequence
  N-terminal Signal Peptide
MVSNKRVLALSALFGCCSLASA

SEQ ID NO: 73 - protein, 20 aa, Artificial Sequence
  N-terminal Signal Peptide
MVSFKSALFAAAAVATVADA

SEQ ID NO: 74 - protein, 24 aa, Artificial Sequence
  N-terminal Signal Peptide
MQKKTAIAIAAGTAIATVAAGTQA

SEQ ID NO: 75 - protein, 18 aa, Artificial Sequence
  N-terminal Signal Peptide
MVSFSSLLAAASLAVVNA

SEQ ID NO: 76 - protein, 18 aa, Artificial Sequence
  N-terminal Signal Peptide
MKNFATLSAVLAGATALA

SEQ ID NO: 77 - protein, 23 aa, Artificial Sequence
  N-terminal Signal Peptide
MKLNKLLSIAAGCTVLGSTYALA

SEQ ID NO: 78 - protein, 21 aa, Artificial Sequence
  N-terminal Signal Peptide
MKLKKLGVILAICLGISSTFA

SEQ ID NO: 79 - protein, 19 aa, Artificial Sequence
  N-terminal Signal Peptide
MKKLLLAACVLFSLASVSA

SEQ ID NO: 80 - protein, 21 aa, Artificial Sequence
  N-terminal Signal Peptide
MIRLKRLLAGLLLPLFVTAFG

SEQ ID NO: 81 - protein, 23 aa, Artificial Sequence
  N-terminal Signal Peptide
MTRSLFIFSLLALAIFSGVSASA

SEQ ID NO: 82 - protein, 24 aa, Artificial Sequence
  N-terminal Signal Peptide
MKLIPNKKTLIAGILAISTSFAYS

SEQ ID NO: 83 - protein, 20 aa, Artificial Sequence
  N-terminal Signal Peptide
MLKRFVKLAVIALAFAYVSA

SEQ ID NO: 84 - protein, 26 aa, Artificial Sequence
  N-terminal Signal Peptide
MKKTGFIGKTLALVIAAGMAGTAAFA

SEQ ID NO: 85 - protein, 21 aa, Artificial Sequence
  N-terminal Signal Peptide
MKLGKLLASVAATLGVSGVNA

SEQ ID NO: 86 - protein, 17 aa, Artificial Sequence
  N-terminal Signal Peptide
MKKLLILACLLISSLES

SEQ ID NO: 87 - protein, 18 aa, Artificial Sequence
  N-terminal Signal Peptide
MTKFLLSLIFITIASALA

SEQ ID NO: 88 - protein, 16 aa, Artificial Sequence
  N-terminal Signal Peptide
MKKTILALALLGSLAA

SEQ ID NO: 89 - protein, 20 aa, Artificial Sequence
  N-terminal Signal Peptide
MRSLGFTFLISALFGVSLSA

SEQ ID NO: 90 - protein, 21 aa, Artificial Sequence
  N-terminal Signal Peptide
MKPACRLISLLMLAVSGIASA

SEQ ID NO: 91 - protein, 17 aa, Artificial Sequence
  N-terminal Signal Peptide
MMLTFFISLLFLSSALA

SEQ ID NO: 92 - protein, 21 aa, Artificial Sequence
  N-terminal Signal Peptide
MTLKTTITLFFAALSANAAFA

SEQ ID NO: 93 - protein, 20 aa, Artificial Sequence
  N-terminal Signal Peptide
MRAKALAASLAGALAGAASA

SEQ ID NO: 94 - protein, 19 aa, Artificial Sequence
  N-terminal Signal Peptide
MVSLSFSLVASAVTHVASA

SEQ ID NO: 95 - protein, 17 aa, Artificial Sequence
  N-terminal Signal Peptide
MVSFSSLNALFLATVLA

SEQ ID NO: 96 - protein, 17 aa, Artificial Sequence
  N-terminal Signal Peptide
MKFQDLTLVLSLSTALA

SEQ ID NO: 97 - protein, 22 aa, Artificial Sequence
  N-terminal Signal Peptide
MRVLSATAFLALLAFGLSGATA

SEQ ID NO: 98 - protein, 21 aa, Artificial Sequence
  N-terminal Signal Peptide
MKFLSTAFVLLIALVAGCSTA

SEQ ID NO: 99 - protein, 20 aa, Artificial Sequence
  N-terminal Signal Peptide
MLKRFLTLFLGFLALASSLA

SEQ ID NO: 100 - protein, 16 aa, Artificial Sequence
  N-terminal Signal Peptide
MKLLTSFVLIGALAFA

SEQ ID NO: 101 - protein, 26 aa, Artificial Sequence
  N-terminal Signal Peptide
MLKKLAMAVGAMLTSISFLLPSSAQA

SEQ ID NO: 102 - protein, 19 aa, Artificial Sequence
  N-terminal Signal Peptide
MKKLLVIAALACGVATAQA

SEQ ID NO: 103 - protein, 19 aa, Artificial Sequence
  N-terminal Signal Peptide
MIKTLLVSSILIPCLATGA

SEQ ID NO: 104 - protein, 23 aa, Artificial Sequence
  N-terminal Signal Peptide
MGIQKKVSILVAGLFMATAFATA

SEQ ID NO: 105 - protein, 16 aa, Artificial Sequence
  N-terminal Signal Peptide
MKKIVALFLVFCFLAG

SEQ ID NO: 106 - protein, 23 aa, Artificial Sequence
  N-terminal Signal Peptide
MNKKVLAAIVLGMLSVFTSAAQA

SEQ ID NO: 107 - protein, 18 aa, Artificial Sequence
  N-terminal Signal Peptide
MKKTAIASALLALPFVFA

SEQ ID NO: 108 - protein, 21 aa, Artificial Sequence
  N-terminal Signal Peptide
MKKTAAIAALAGLSFAGMAHA

SEQ ID NO: 109 - protein, 19 aa, Artificial Sequence
  N-terminal Signal Peptide
MISANKILFLILCVACVSA

SEQ ID NO: 110 - protein, 18 aa, Artificial Sequence
  N-terminal Signal Peptide
MVKLASILLIILAGESFA

SEQ ID NO: 111 - protein, 18 aa, Artificial Sequence
  N-terminal Signal Peptide
MINKLIALTVLFSLGINA

SEQ ID NO: 112 - protein, 24 aa, Artificial Sequence
  N-terminal Signal Peptide
MVASLWSSILPVLAFLWADLSAGA

SEQ ID NO: 113 - protein, 17 aa, Artificial Sequence
  N-terminal Signal Peptide
MKFLLFIALSLAVATAA

SEQ ID NO: 114 - protein, 21 aa, Artificial Sequence
  N-terminal Signal Peptide
MRHFLSLLLYGATLVSSSACS

SEQ ID NO: 115 - protein, 18 aa, Artificial Sequence
  N-terminal Signal Peptide
MKFSAIVLLAALAFAVSA

SEQ ID NO: 116 - protein, 20 aa, Artificial Sequence
  N-terminal Signal Peptide
MKKRLLIASVALGSLFSFCA

SEQ ID NO: 117 - protein, 20 aa, Artificial Sequence
  N-terminal Signal Peptide
MSWRSIFLLVLLASIDFING

SEQ ID NO: 118 - protein, 15 aa, Artificial Sequence
  N-terminal Signal Peptide
MRLPSLLLPLAALIA

SEQ ID NO: 119 - protein, 17 aa, Artificial Sequence
  N-terminal Signal Peptide
MKVLAALVLALVATASA

SEQ ID NO: 120 - protein, 4 aa, Artificial Sequence
  N-terminal Signal Peptide
MARA

SEQ ID NO: 121 - protein, 18 aa, Artificial Sequence
  N-terminal Signal Peptide
MRKLLIWLAGFLVLILKT

SEQ ID NO: 122 - protein, 21 aa, Artificial Sequence
  N-terminal Signal Peptide
MRKFISSLLLGLVVSIATAVA

SEQ ID NO: 123 - protein, 20 aa, Artificial Sequence
  N-terminal Signal Peptide
MNTLFLFTSLFLFLFAKVTA

SEQ ID NO: 124 - protein, 19 aa, Artificial Sequence
  N-terminal Signal Peptide
MKFLILLITLGAIAATALA

SEQ ID NO: 125 - protein, 25 aa, Artificial Sequence
  N-terminal Signal Peptide
MRVTSKVILTLIAATAFATAFTWSA

SEQ ID NO: 126 - protein, 21 aa, Artificial Sequence
  N-terminal Signal Peptide
MKKFKRTILSGLALAMSIAQA

SEQ ID NO: 127 - protein, 19 aa, Artificial Sequence
  N-terminal Signal Peptide
MLFKSVLLALASAGVAVNA

SEQ ID NO: 128 - protein, 20 aa, Artificial Sequence
  N-terminal Signal Peptide
MKLFKILTACLFIGLLNVSA

SEQ ID NO: 129 - protein, 15 aa, Artificial Sequence
  N-terminal Signal Peptide
MAVMRFFASLPRRVA

SEQ ID NO: 130 - protein, 24 aa, Artificial Sequence
  N-terminal Signal Peptide
MLKRAAFLVGVSLAVAAGCGPAQA

SEQ ID NO: 131 - protein, 23 aa, Artificial Sequence
  N-terminal Signal Peptide
MTHRTFAALPAAALAAVSSAAFA

SEQ ID NO: 132 - protein, 21 aa, Artificial Sequence
  N-terminal Signal Peptide
MKLSQSLTYLAVLGLAAGANA

SEQ ID NO: 133 - protein, 17 aa, Artificial Sequence
  N-terminal Signal Peptide
MASKLAFFLALALAAAA

SEQ ID NO: 134 - protein, 21 aa, Artificial Sequence
  N-terminal Signal Peptide
MKFLSLKLVVLAFYVAFQINA

SEQ ID NO: 135 - protein, 15 aa, Artificial Sequence
  N-terminal Signal Peptide
MAKLIALVLLGLAAA

SEQ ID NO: 136 - protein, 15 aa, Artificial Sequence
  N-terminal Signal Peptide
MRSLLLTLLGALLRA

SEQ ID NO: 137 - protein, 20 aa, Artificial Sequence
  N-terminal Signal Peptide
MKLNIVKLLVLAAFAQAASA

SEQ ID NO: 138 - protein, 16 aa, Artificial Sequence
  N-terminal Signal Peptide
MILFYVLPVVLALVSG

SEQ ID NO: 139 - protein, 20 aa, Artificial Sequence
  N-terminal Signal Peptide
MKKNLLKLTLALISGMSQFA

SEQ ID NO: 140 - protein, 19 aa, Artificial Sequence
  N-terminal Signal Peptide
MKFLIPLFVLFIVFGNAYA

SEQ ID NO: 141 - protein, 19 aa, Artificial Sequence
  N-terminal Signal Peptide
MKRVFSLFTAVCGLLSVSA

SEQ ID NO: 142 - protein, 16 aa, Artificial Sequence
  N-terminal Signal Peptide
MKKFSIFLVLSITVLA

SEQ ID NO: 143 - protein, 16 aa, Artificial Sequence
  N-terminal Signal Peptide
MKKKIVAVLTLSVVLA

SEQ ID NO: 144 - protein, 24 aa, Artificial Sequence
  N-terminal Signal Peptide
MKKRVISALAALWLSVLGAPAVLA

SEQ ID NO: 145 - protein, 22 aa, Artificial Sequence
  N-terminal Signal Peptide
MGVFSFLTTEAMAVFLAGLAHA

SEQ ID NO: 146 - protein, 21 aa, Artificial Sequence
  N-terminal Signal Peptide
MTMKGLRVVALVVLASLGIFA

SEQ ID NO: 147 - protein, 22 aa, Artificial Sequence
  N-terminal Signal Peptide
MTKFLSASLALLSGLATASSDA

SEQ ID NO: 148 - protein, 20 aa, Artificial Sequence
  N-terminal Signal Peptide
MTQKSLLLALTAVALVSVNA

SEQ ID NO: 149 - protein, 19 aa, Artificial Sequence
  N-terminal Signal Peptide
MNRLYAVFAVLCFAQVLHG

SEQ ID NO: 150 - protein, 19 aa, Artificial Sequence
  N-terminal Signal Peptide
MKKLLLQSLILSELGGCLA

SEQ ID NO: 151 - protein, 19 aa, Artificial Sequence
  N-terminal Signal Peptide
MAARSVLLLALLTLAVSTA

SEQ ID NO: 152 - protein, 19 aa, Artificial Sequence
  N-terminal Signal Peptide
MKGTLAFLLVFLLNLYVHG

SEQ ID NO: 153 - protein, 48 aa, Artificial Sequence
  N-terminal Signal Peptide
MLSIDTSSTRRVVPNTALFPNTHRRDFATAGQLLAMASAVLTGAPAHA

SEQ ID NO: 154 - protein, 21 aa, Artificial Sequence
  N-terminal Signal Peptide
MNISIFVGKLALAALGSALVA

SEQ ID NO: 155 - protein, 18 aa, Artificial Sequence
  N-terminal Signal Peptide
MRRLFLLSSLASLSVASA

SEQ ID NO: 156 - protein, 28 aa, Artificial Sequence
  N-terminal Signal Peptide
MKCCRIMFVLLGLWFVFGLSVPGGRTEA

SEQ ID NO: 157 - protein, 17 aa, Artificial Sequence
  N-terminal Signal Peptide
MKFLILATLSIFTGILA

SEQ ID NO: 158 - protein, 17 aa, Artificial Sequence
  N-terminal Signal Peptide
MKVFTLAFFLAIIVSQA

SEQ ID NO: 159 - protein, 18 aa, Artificial Sequence
  N-terminal Signal Peptide
MKKKIAITLLFLSLLNRA

SEQ ID NO: 160 - protein, 20 aa, Artificial Sequence
  N-terminal Signal Peptide
MKLLKVIATAFLGLTSFASA

SEQ ID NO: 161 - protein, 20 aa, Artificial Sequence
  N-terminal Signal Peptide
MPTVVALDLATYVLQPSKRA

SEQ ID NO: 162 - protein, 16 aa, Artificial Sequence
  N-terminal Signal Peptide
MLMVPLLLALGAVAAG

SEQ ID NO: 163 - protein, 26 aa, Artificial Sequence
  N-terminal Signal Peptide
MPAARRLALFAAVALAAVGLSPAALA

SEQ ID NO: 164 - protein, 21 aa, Artificial Sequence
  N-terminal Signal Peptide
MRSLLLTSALAALVSLAAASA

SEQ ID NO: 165 - protein, 512 aa, Artificial Sequence
  N-terminal Signal Peptide
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 166 - protein, 501 aa, Artificial Sequence
  N-terminal Signal Peptide
MRRWVVAMLAVLFLFPSVVVADGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 167 - protein, 512 aa, Artificial Sequence
  N-terminal Signal Peptide
MKQHKRLYARLLPLLFALIFLLSHSAAAAASLNGTLMQYFEWYMPNDGQHWKRLQNDSAYLAEHGITAVWIPPAYKGTSQDDVGYGAYDLYDLGEFHQKGTVRTKYGTKGELQSAINSLHSRDINVYGDVVINHKGGADATEDVTAVEVDPADRNRVTSGEQRIKAWTHFQFPGRGSTYSDFKWYWYHFDGTDWDESRKLNRIYKFQGKAWDWEVSNENGNYDYLMYADIDYDHPDVTAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPVKAVTFVDNHDTQPGQSLESTVQTWFKPLAYAFILTREAGYPQIFYGDMYGTKGASQREIPAMKHKIEPILKARKQYAYGAQHDYFDHHNIVGWTREGDSSVANSGLAALITDGPGGTKRMYVGRQNAGETWDDITGNRSDSVVINAEGWGEFHVNGGSVSIYVQR

SEQ ID NO: 168 - protein, 477 aa, Artificial Sequence
  N-terminal Signal Peptide
MFAKRFKTSLLPLFAGFLLLFYLVLAGPAAASAETANKSNELTAPSIKSGTILHAWNWSFNTLKLNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLERALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQHEELSNPNGNNQIFMNQRGSHGVVLANAGSSSVSINTATKLPDGRYDNKAGAGSFQVNDGKLTGTINARSVAVLYPDDIEIRCNTFFQ

SEQ ID NO: 169 - protein, 660 aa, Artificial Sequence
  N-terminal Signal Peptide
MAFAKRFKTSLLPLFAGFLLLFHLVLAGPTAANAETANKSNELTAPSIKSGTILHAWNWSFNTLKHNMKDIHDAGYTAIQTSPINQVKEGNQGNKSMSNWYWLYQPTSYQIGNRYLGTEQEFKEMCAAAEEYGIKVIVDAVINHTTSDYAVISNEIKSIPNWTHGNTQIKNWSDRWDVTQNSLLGLYDWNTQNTQVQSYLKRFLERALNDGADGFRFDAAKHIELPDDGSYGSQFWPNITNTSAEFQYGEILQDSASRDAAYANYMNVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANTGSSSVSINTPTKLPNGRYDNKAGAGSFQVNDGKLTGTINARSVAVLYPDDIAKAPHVFLENYKTGVTHSFNDQLTITLRADANTTKAVYQINNGPETAFKDGDQFTIGKGDPFGKTYTIMLKGTNSDGVTKTEEYSFVKRDPASAKTIGYQNPNHWSQVNAYIYKHDGGRAIELTGSWPGKPMTKNADGIYTLTLPVDTDTTNAKVIFNNGSAQVPGQNQPGFDYVQNGLYNDSGLSGSLPH

SEQ ID NO: 170 - protein, 487 aa, Artificial Sequence
  N-terminal Signal Peptide
MKAKWILSIISSVLVLTLVNPAFAADERELKDELIYDVLVDRYFNKKIDNDYEVNALDPASFNGGDFDGMASELLFVKEMGFTALSIGPVFSTATYDGKRVLDYTKFERHFGTKEEFEELVAEIHDQDMKVIVDVPTQQVSEEHVWVAENPDWFTENEDGSLALDTSNLEAQEALISTFTEFSETYEIDGFRLQNADQLDESFVSDFSDAIKDVRDSYILSDREMAEMPGVDAVVLPGVEETLRAAYKNFDQDLTGISSIMEESEDNLIQVDSLIGSRFTADIVEENGFPPTRWTLLLTQLLTMPGIPVVQYGSESAMNGTELPESHQILDLAVDKELVDHITDLTSLRNSSEALRTGELEILHDEDGWLVYKRSNDEETWIIAINNSSVTKNINLPAEVIGEGQEMRGLFESDIIRQEDNGEYRLTQDREIAEVFHVTEETKLNSAYIAVLAIMYVVFMLFLWVVWRKGKQRKADAAKQKANEKTV

SEQ ID NO: 171 - protein, 479 aa, Artificial Sequence
  N-terminal Signal Peptide
MKAKWISSMITAVLLLTIVQPAYAEKEYELKDELIYDVLVDRYFNKKIDNDYEVNALDPTTFNGGDFDGMASELLFVKDMGFTALSIGSVFSTETYDGKKVLDYTEFERHFGTKEEFQTLVEEIHEQDMKVIVDIPTQQVSANHIWAVENPQWFTKNEDGSLAIDTSNLEVQKALITTFTEFNETYQVDGFRLQDADKIDSQFVRDFSKAMKDIRPSYILSDREMSEKTGFDAVVIPGVEETLRAAYKNFDQDLTGIKTLMKQSENQLIQVDSLLGSRFTSDIVEVKGFPPTRWKLLLTQLLTMPGIPVIQYGSESAMNGVAPSESHQILDMAVDKELVDHITNLTSLRNSSEALRTGKVEVLHDEDGWLVYKRSNDDETWVIAINNSSSTKTINLSADVIGENQELQGLFESDIVRQEDSGDYRVTQDREIAEVFHVREETKLNTAYIATLAIMYIVFMLFLWFVWRKGKQRKADAAK

SEQ ID NO: 172 - protein, 453 aa, Artificial Sequence
  N-terminal Signal Peptide
MRFALKIGIIFLQLEVWGMRVKKIWTKELFICFCLAVILFVPVHTFAEEKREWRDEVIYSIMIDRFNNGESKNDKQLDVGNLEGYQGGDIRGIIKRLDYIKEMGFTTIMLSPLFESEKYDGLSVRNFQKANEHFGTENDVKELVNEAHKKGMKVVFQFPYEENEQQLIDSMKWWIKEVDLDGSYVIHSEKKPRAFWDEVQKDLQKVKKDFHIMTKEDNEYYKKIVESFSKADMSVKPLYDVSGKEEEFATFLDNQETKRFARIAKENMYYPPSRLKLAITYLLTSPGIPNFYYGTEIALDGGDAPDNRRLMDFKSDEKFMQHITKLGELRQTRPSLRRGTFELLYDKDGMSILKRKYKDEVTLVAINNTKKTQKVSLPVSAIGEKQELRGLLEDEIIREENGKFYLVLKREESNVYKVSEETGVNWLFISLIVGVNVLFIAFLIAVKKKRSSV

SEQ ID NO: 173 - protein, 500 aa, Artificial Sequence
  N-terminal Signal Peptide
MLKKLIVILLVPLLLFPLFSKPGSAADWQDDAIYYVMVDRFYNGNTQNDQEVNIDDMNTYQGGDFAGITEKLDYIKEMGFTAIALNPVIQNMNGDYTGGSPHDFTAVNENYGTMEELRKLVKEAHDRDMKIMLDFQVNHVGEGHPWLEENGKKDWFHEERAITENGQTDKQETGWLDGKPDLAQENPEVSSYLIDAAKWWIKETGIDGYRLIYMNYVPQSFWEEMISAIREDRPDFFFIGDVDADKAEKLGIGMLDEEQMTGMREAFSTTDVEMDSLFKRWETLFGALNTPYTTGISVDTERTERFTKETERAKQFPGTRWKMALTYMYTMPGIPVVTYGSEIALNGGEKPDNHRMMNFRTDEELIDYMKQLSALRKEYPALRTGDLELLQSEDGMVVFKRETEEQTMIAAINNSSKTKTIQLDSAFIQDNQELRGKLNTDLVREQNGTFSITLEREMAEVYLVEKESGINYGFLAVLILVPIAFILFLILARKRGRQTN

SEQ ID NO: 174 - protein, 499 aa, Artificial Sequence
  N-terminal Signal Peptide
MLDSSIEEDFELNVKWISTAVASILLATAIGAAPAVSAKEETPEIQNESIYDVLIDRFFDKNVENDYNINAKDPKAFHGGDFDGIATKLSYFQDLGFTMLSLGSVFSSETYDGQAVVDYNKIERHFGTGKEFSSLIGTLHKNHIKTMADFPINNVSKNHAWVLEKGHQDWYELANSDRINWNTSNTAVQDALVQAAVDFVGKYKVDGIRLTELGKANTEFLNKMITALKQKNKDLYVITNEPSYADFDLKVNKDTIDTYQSIFKNVDQDSSQLEKPLGNSINDTSQKPVALMLDDLYSARFTHASAEENMFPPTRIKVALGAIMTLPGVPIVSYGTEIAQNGVTTDESHAPMDFRTKDDIISYIEDLQSVRNKSKALRTGKFKLLENKDGLMVYERYAGKEKWIIVVNNTDKTKRYVLDPSVIGNNKELHGIFEKDIVRQQKEDEKYNIVLDRELVEFYQVTDHKGINIPYMIALGLVYVIFITFIVLLLRRGRRGKKA

SEQ ID NO: 175 - protein, 549 aa, Artificial Sequence
  N-terminal Signal Peptide
MLTFHRIIRKGWMFLLAFLLTALLFCPTGQPAKAAAPFNGTMMQYFEWYLSCDPQSFSEPPQCEVKVANLGITALWLPPAYKGTSRSDVGYGVYDLYDLGEFNQKGAVRTKYGTKAQYLQAIQAAHAAGMQVYADVVFNHKGGADGTEWVLQISWWVDAVNQEISGTYQIQAWTKFDFPGRGNTYSSFKWRWYHFDGVDWDESRKLSRIYKFRGIGKAWDFHFAYFEKAYYDSTENGDIDDHPEVVTELKSWGKWYVNTTNIDGFRLDAVKHIDFSFFPDWLSDVRSQTGKPLFTVGEYWSYDINKLHNYIMKTNGTMSLFDAPLHFHDRTASKSGDAYDMRTLMTNTLMKDQPTLAVTFVDNHDTEPGQALQSWVDPWFKPLAYAFILTVIDRRRDGYGGDYYGIPQYNPSLKSKIDPLLIARRDYAYGTGHDYLDHSHDIIGWTREGVTEKPGSGLAALITDGPGGSKWMYVGKQHAGGNRTTVGGMETALTNGSSCDRQEGYPCVFYVSVWVPRKTTVSTIAWSITTRPWTDEFVRWTEPRLVAWP

SEQ ID NO: 176 - protein, 549 aa, Artificial Sequence
  N-terminal Signal Peptide
MLTFHRIIRKGWMFLLAFLLTASLFCPTGQPAKAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEGVDAVEVNPSDCNQEFSGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFGDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTITRPITTRPWTGEFVRWTEPRLVAWP

SEQ ID NO: 177 - protein, 596 aa, Artificial Sequence
  N-terminal Signal Peptide
MIRSLLLALTLGASALPSAVAAQDFRARAPEDEVIYFVLPDRFENGDKANDRGGMTGDRLAHGFDPTAKGFYHGGDLKGLMARLPYIQSLGATAIWVGPIFRNKAVQGGPGNESAGYHGYWVTDFTRVDPHLGTNADFKALVDAAHARGMKVYMDIIANHTADVIQYRECAGKRECAYRSRADYPYAAKGKNKGFAGDGVQTAENFAKLTDPSFAYTPFVPAAERSVKAPAWLNDPIYYHNRGNTTFSNESSQMGDFVGLDDLMTENPRVLAGMIDIFGGWIDRFGIDGYRIDTARHVNPEFWAAFVPAMQARAKARGIPNFHVFGEVALSSLDAGGLAVWTKTGKFPAVLDFSFREAVLQSVAGTKGTDVFETVFDGDVLYAGGEATARQLPTFTGNHDNGRFAFYLRQARPQASDAEILSRVMLSNAMLLSLRGVPTIYSGDEQGFAGDGNDQDAREDMFASQVASYNDNKLVGTTATTATANFAPTHPLYRQIAQLAKLRTSTPALTRGRQLLRAREDKPGLLAISRFDPTSNAEVLLLFNTANAAITRNVQVETASTRFRALTGRCGTVTAPGAIRVDLPALGYAVCAAETR

SEQ ID NO: 178 - protein, 545 aa, Artificial Sequence
  N-terminal Signal Peptide
MKQLCKKGLAFVLMFIFVNAFILSPINGAAAVDGKSMNPGYKTYLMAPLKKVTDYTTWEAFENDLRKAKQNGFYAVTVDFWWGDMEKNGDQQFDFSYAQRFAQAARNAGIKIVPIISTHQCGGNVGDDCNVPLPSWVWNLKSDDSLYFKSETGTTNKETLSPLATDVISKQYGELYTAFAQALAPYKDVIAKIYLSGGPAGEIRYPSYTAADGTGYPSRGKFQVYTNFAKSKFQSYALTKYGSLAGINQAWGTNLTSASQILPPSDGYQFLKDGYSTAYGKDFLAWYQGALEDHTKRIGQLAHQAFDSTFNVPIGAKVAGIHWQYNNPTIPHAAEKPAGYNDYNALLDAFKTAKLDITFTCLEMTDSGNYPEYSMPKTLVRQVAGIANAKGVVLNGENALTIGSEDQYKKAAEMAFNYNFAGFTLLRFYDVINNDTLMGQFKNTLGVTPLAQTVVVKNAPTALGETVYIVGDRAELGQWDTSIYPIKLTYNSSTADWRGTVYFPASQNVQFKAIVKRADGSLKAWQPSQQYWSVPSTTTTYTDNW

SEQ ID NO: 179 - protein, 516 aa, Artificial Sequence
  N-terminal Signal Peptide
MKLHNRIISVLLTLLLAVAVLFPYMTEPAQAHHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 180 - protein, 288 aa, Artificial Sequence
  N-terminal Signal Peptide
MSPNPTNIHTGKTLRLLYHPASQPCRSAHQFMYEIDVPFEEEVVDISTDITERQEFRDKYNPTGQVPILVDGEFTVWESVAIARYVNEKFDGAGNWFGRGTQERAQINQFLQWYAYTLRLGGGAFHWNIFGCLIYGEKPYSPKFTAEQNKGRTLLYEAMGTLENYWLRDREYVCGDEVSYADLAAFHEFVSHEAGKIIPDRVWQGFPKIAAWFKKLSERPHAKTVSEWQYTNVGKIIRGELTASMFKRKTAVLKGTEVFSGHNHGIPYLNEKAEDYFKRVEKEGAAVA

SEQ ID NO: 181 - protein, 342 aa, Artificial Sequence
  N-terminal Signal Peptide
MPPGCSGLCSFVGLTMPLKFSGVLCASLLTITLSVAAHATELILDFNKVQMRSQQLAPGVYAHLPADSAELNAKGGVAGTSGGLIVGTRGAMLIETMLNRRLFDQVQALAKKEALGLPLLYAVNTSYHGDHSYGNMYLKAPTRVIQSTKTRDYVDGHLADDKAFMVKNFGAGRGVEQITARTGDILVPPGGRVSVDLGGKTVEIIDFGFAQTGGDLFVWEPQSKVMWTGNAVVASKPALPWLLDGKLVETLATLQKVYDFLPPDATIVPGHGVPMAREGLRWHLDYLAAVQAGVKDALARKLSLEQTVTELKMPEFRGYVLFDWVHPDLNVPAAYKDLAARI

SEQ ID NO: 182 - protein, 306 aa, Artificial Sequence
  N-terminal Signal Peptide
MTYTEIVTGSTPDDRFDNLAGYPSAPHYVDVTAGDTGPLRMHYVDEGPRDGTPVVLLHGEPTWSYLYRTMIPPLAAGGCRVLAPDLIGFGRSDKPSRIEDYTYLRHVEWVTSWFEALDLSDTTIFVQDWGSLIGLRIAAEQGVRVGRIVVANGFLPAGQTSANLPFRLWRAFARYTPVLPAGRIVAFGTVTDVPARVRAGYDAPFPGKRFQAGARAFPALVPTSPDDPALPANRAAWEALGRWEKPFLCVFGAKDPILGRADRPLIEHVPGAAGQPHARINGSHFVQEDCGPELAERILQWSTNPA

SEQ ID NO: 183 - protein, 319 aa, Artificial Sequence
  N-terminal Signal Peptide
MAGNITPTLANWSTWETFKAAQQEIVLPAGRVVHIAVTIRYVDIGEPRWGTILLMHGIPTWGCLYHAIIPPLAQAGYRVIAPDFLGHGWSDRRDRFDRSFQDQARMVTALLDTLQLGSVDVVGHDTGGAVALILAIEHKVRVNRLVICNSVCSDRFDDDMLDFGHPLRWKPRPVADLVAVLEESLAAGLSNQNRLTLEFRAGIIAPWASEEGKLSLLRNASALNANQTVALVDRHGEITAPTLVLWGMDDPWQRADDGKRLAREIPRAVFQAIEGASHWVQQGARAIHRCPAGFLRQCAGFCRVAEWERIWSTVLWPCR

SEQ ID NO: 184 - protein, 302 aa, Artificial Sequence
  N-terminal Signal Peptide
MDVLRTPDERFEGLADWSFAPHYTEVTDADGTALRIHHVDEGPKDQRPILLMHGEPSWAYLYRKVIAELVAKGHRVVAPDLVGFGRSDKPAKRTDYTYERHVAWMSAWLEQNDLKDIVLFCQDWGGLIGLRLVAAFPERFSAVVVSNTGLPIGVGKSEGFEAWLNFSQNTPELPVGFILNGGTARDLSDAERSAYDAPFPDESYKEGARIFPALVPITPEHASVEENKAAWAVLETFDKPFVTAFSDADPITRGGEAMFLARVPGTKNVAHTTLKGGHFVQEDSPVEIAALLDGLVAGLPQA

SEQ ID NO: 185 - protein, 307 aa, Artificial Sequence
  N-terminal Signal Peptide
MSEIGTGFPFDPHYVEVLGERMHYVDVGPRDGTPVLFLHGNPTSSYLWRNIIPHVAPSHRCIAPDLIGMGKSDKPDLDYFFDDHVRYLDAFIEALGLEEVVLVIHDWGSALGFHWAKRNPERVKGIACMEFIRPIPTWDEWPEFARETFQAFRTADVGRELIIDQNAFIEGALPKFVVRPLTEVEMDHYREPFLKPVDREPLWRFPNELPIAGEPANIVALVEAYMNWLHQSPVPKLLFWGTPGVLISPAEAARLAESLPNCKTVDIGPGLHFLQEDNPDLIGSEIARWLPALIVGKSIEFDGGWAT

SEQ ID NO: 186 - protein, 290 aa, Artificial Sequence
  N-terminal Signal Peptide
MENMSTTYPIVLVHGLSGFDDIVGYPYFYGIRDALEKDGHKVFTASLSAFNSNEVRGEQLWEFVQKVLKETKAKKVNLIGHSQGPLACRYVAAKHAKNIASVTSVNGVNHGSEIADLVRRIMRKDSVPEYIADAVMKAIGTIISTFSGNRGNPQDAIAALEALTTENVMEFNKKYPQGLPAIRGGEGKEVVNGVHYYSFGSYIQGLIAGEKGNLLDPTHAAMRVLSAFFTERENDGLVGRTSMRLGKLIKDDYAEDHLDMVNQVAGLVGPGEDIVAIYTNHANFLASKKL

SEQ ID NO: 187 - protein, 416 aa, Artificial Sequence
  N-terminal Signal Peptide
MKCCRVMFVLLGLWLVFGLSVPGGRAEAATSRANDAPIVLLHGFTGWGREEMFGFKYWGGVRGDIEQWLNDNGYRTYTLAVGPLSSNWDRACEAYAQLVGGTVDYGAAHAAKHGHARFGRTYPGLLPELKRGGRIHIIAHSQGGQTARMLVSLLENGSQEEREYAKAHNVSLSPLFEGGHHFVLSVTTIATPHDGTTLVNMVDFTDRFFDLQKAVLEAAAVASNAPYTSEIYDFKLDQWGLRREPGESFDHYFERLKRSPVWTSTDTARYDLSVPGAETLNRWVKASPNTYYLSFSTERTYRGALTGNYYPEFGMNAFSAIVCAPFFGSYRNAALGIDSHWLENDGIVNTISMNGPKRGSSDRIVPYDGALKKGVWNDMGTYNVDHLEIIGVDPNPSFDIRAFYLRLAEQLASLRP

SEQ ID NO: 188 - protein, 212 aa, Artificial Sequence [sequence omitted]

SEQ ID NO: 189 - protein, 210 aa, Artificial Sequence
  N-terminal Signal Peptide
MKKVLMAFIICLSLILSVLAAPPSGAKAESVHNPVVLVHGISGASYNFFAIKNYLISQGWQSNKLYAIDFYDKTGNNLNNGPQLASYVDRVLKETGAKKVDIVAHSMGGANTLYYIKYLGGGNKIQNVVTLGGANGLVSSTALPGTDPNQKILYTSIYSLNDQIVINSLSRLQGARNIQLYGIGHIGLLSNSQVNGYIKEGLNGGGLNTN

SEQ ID NO: 190 - protein, 291 aa, Artificial Sequence
  N-terminal Signal Peptide
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 191 - protein, 313 aa, Artificial Sequence
  N-terminal Signal Peptide
MNNKKTLLALCVGSSLLLSGQADAGLFGSTGYTKTKYPIVLTHGMLGFDSILGVDYWYGIPSSLRSDGASVYITEVSQLNTSELRGEELLDQVEEIAAISGKGKVNLIGHSHGGPTVRYVAAIRPDLVASVTSVGAPHKGSDTADFLRQIPPGSAGEALVAGIVNGLGTLINFLSGSPSTTPQNALGALESLNSQGAAVFNAKYPQGIPTSACGEGAYKVNGVSYYSWSGTSPLTNILDVSDLLMGASSLTFDEANDGLVGRCSSHLGKVIRDNYRMNHLDEVNQVFGLTSLFETDPVTVYRQQANRLKLAGL

SEQ ID NO: 192 - protein, 311 aa, Artificial Sequence
  N-terminal Signal Peptide
MNKNKTLLALCLGSALALSGQAFAATGSGYTATKYPIVLAHGMLGFDSLLGIDYWYGIPSALRRDGAQVYVTEVSQLNTSELRGEELLAQVEEIVAISGKPKVNLIGHSHGGPTIRYVAGVRPDLIASVTSVGAPHKGSDVADLIRKVPEGSSGEAIIAGLVNAMGAFINFVSGSSSTAPQNSLGSLESLNSEGAARFNAKFPQGIPTTACGEGAYKVNGVHYYSWSGTSPLTNPLDVSDAMMGAGSLAFSGPNDGLVGRCSSHLGMVIRDNYRMNHLDEVNQFMGLTSLFETDPVSVYRQHANRLKNAGL

SEQ ID NO: 193 - protein, 291 aa, Artificial Sequence
  N-terminal Signal Peptide
MRSSLVLFFVSAWTALASPVRREVSQDLFDQFNLFAQYSAAAYCAKNNDAPAGANVTCRGSICPEVEKADATFLYSFEDSGVGDVTGFLALDNTNRLIVLSFRGSRSLENWIGNINLDLKGIDDICSGCKGHDGFTSSWRSVANTLTQQVQNAVREHPDYRVVFTGHSLGGALATVAGASLRGNGYDIDVFSYGAPRVGNRAFAEFLTAQTGGTLYRITHTNDIVPRLPPRELGYSHSSPEYWITSGTLVPVTKNDIVKVEGIDSTDGNNQPNTPDIAAHLWYFGLIGTCL

SEQ ID NO: 194 - protein, 223 aa, Artificial Sequence
  N-terminal Signal Peptide
MTTAMPSDAAIVALCAQIYQPSAPDAFEYFDAGTDDGICWAIKRLNGLDVVVLRGSRTLQDWLRDIHAFPAPSRIGHVHSGFYAGMEHMWSDLKRLLEQPAIVTGHSLGAARAAVLTALMTVDHVPPVARVVFGEPKPGLLDFAQLITRIPGRSYRNGDDTHHDLVTDVPFSFPPMQYVHPTPIIPVCAEPSGDLFSTMGVFAYHHVELYQAALAVQPQEKVA

SEQ ID NO: 195 - protein, 254 aa, Artificial Sequence
  N-terminal Signal Peptide
MAFDALFARDVALPLAAAAYSVLGGSPAVLPTGFVKTALIRADGAALTAMTDPHPAVTAMTKDTDLFGLLGHNPASRTAFVSFRGSADLADWLADIDALPQPYLPIAGFGHVHAGFQQVYELVRDSVAANLAAATAGTDQILVTGHSLGAALAVLAAPDVCRNMPPNTIEPRLVTFAGPRVGLSDFAAAFNAAIESCYRVVNFLDIVPHVPPSPYVHVGVEIDVDSGGAIDVAWRHSLTAYRDGLTALIAAASA

SEQ ID NO: 196 - protein, 375 aa, Artificial Sequence
  N-terminal Signal Peptide
MVGLVCVTALVTVTDSASAAEEKVKYLIGFEEEAELEAFTEEIDQVGVFSVEEQSVAEDTLDIDVDIIDEYDYTDVLAVELDPEDVDALSEEAGISFIEEDIELSIQQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 197 - protein, 381 aa, Artificial Sequence
  N-terminal Signal Peptide
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 198 - protein, 382 aa, Artificial Sequence
  N-terminal Signal Peptide
MRGKKVWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAYAQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYNGTSMASPHVAGAAALILSKHPNWTNTQVRSSLENTTTKLGDSFYYGKGLINVQAAAQ

SEQ ID NO: 199 - protein, 379 aa, Artificial Sequence
  N-terminal Signal Peptide
MMRKKSFWLGMLTAFMLVFTMAFSDSASAAQPAKNVEKDYIVGFKSGVKTASVKKDIIKESGGKVDKQFRIINAAKAKLDKEALKEAKNDPDVAYVEEDHVAHALAQTVPYGIPLIKADKLHAQGFKGANVKGAVLATGIPTSHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 200 - protein, 380 aa, Artificial Sequence
  N-terminal Signal Peptide
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 201 - protein, 213 aa, Artificial Sequence
  N-terminal Signal Peptide
MFKFKKNFLVGLSAALMSISLFSATASAASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQTKRPTGSNATITFSNHVNAWKSHGMNLGSNWAHQVMATEGYQSSGSSNVTVW

SEQ ID NO: 202 - protein, 329 aa, Artificial Sequence
  N-terminal Signal Peptide
MVQLKTAALALLFAGQAISSPVDIDSRQASVSIDAKFKAHGKKYLGTIGDQYTLTKNSKNPAIIKADFGQLTPENSMKWDATEPNRGQFSFSGSDYLVNFAQSNGKLIRGHTLVWHSQLPGWVSSITDKNTLISVLKNHITTVMTRYKGKIYAWDVLNEIFNEDGSLRNSVFYNVIGEDYVRIAFETARSVDPNAKLYINDYNLDSAGYSKVNGMVSHVKKWLAAGIPIDGIGSQTHLGAGAGANVAGALNALAGAGTTEIAITELDIAGASSTDYVNVVKACLNQSKCVGITVWGVADPDSWRSSSSPLLFDSNYNPKAAYNAIANAL

SEQ ID NO: 203 - protein, 228 aa, Artificial Sequence
  N-terminal Signal Peptide
MNLRKLRLLFVMCIGLTLILTAVPAHARTITNNEMGNHSGYDYELWKDYGNTSMTLNNGGAFSAGWNNIGNALFRKGKKFDSTRTHHQLGNISINYNASFNPGGNSYLCVYGWTQSPLAEYYIVDSWGTYRPTGAYKGSFYADGGTYDIYETTRVNQPSIIGIATFKQYWSVRQTKRTSGTVSVSAHFRKWESLGMPMGKMYETAFTVEGYQSSGSANVMTNQLFIGN

SEQ ID NO: 204 - protein, 248 aa, Artificial Sequence
  N-terminal Signal Peptide
MTVRPEYNLIKREECQMKQKRMKFLLAVLFSLALTLPAWSAQAATTITSNQTGTQDGYDYELWKDSGNTSMTLNSGGAFSAQWSNIGNALFRKGKKFDSTKTHSQLGNISINYNATFNPGGNSYLCVYGWTKDPLTEYYIVDNWGTYRPTGTPKGTFTVDGGTYDIYETTRINQPSIIGIATFKQYWSVRQTKRTSGTVSVSEHFKKWESLGMPMGKMYETALTVEGYQSNGSANVTANVLAISGKSF

SEQ ID NO: 205 - protein, 223 aa, Artificial Sequence
  N-terminal Signal Peptide
MKLSLAAIGICTTAAVAFPSGLTQHATGDLSKRQSITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 206 - protein, 442 aa, Artificial Sequence
  N-terminal Signal Peptide
AERQPLKIPPIIDVGRGRPVRLDLRPAQTQFDKGKLVDVWGVNGQYLAPTVRVKSDDFVKLTYVNNLPQTVTMNIQGLLAPTDMIGSIHRKLEAKSSWSPIISIHQPACTCWYHADTMLNSAFQIYRGLAGMWIIEDEQSKKANLPNKYGVNDIPLILQDQQLNKQGVQVLDANQKQFFGKRLFVNGQESAYHQVARGWVRLRIVNASLSRPYQLRLDNDQPLHLIATGVGMLAEPVPLESITLAPSERVEVLVELNEGKTVSLISGQKRDIFYQAKNLFSDDNELTDNVILELRPEGMAAVFSNKPSLPPFATEDFQLKIAEERRLIIRPFDRLINQKRFDPKRIDFNVKQGNVERWYITSDEAVGFTLQGAKFLIETRNRQRLPHKQPAWHDTVWLEKNQEVTLLVRFDHQASAQLPFTFGVSDFMLRDRGAMGQFIVTE

SEQ ID NO: 207 - protein, 28 aa, Artificial Sequence
  N-terminal Signal Peptide
MMNLTRRQLLTRSAVAATMFSAPKTLWA

SEQ ID NO: 208 - protein, 344 aa, Artificial Sequence
  N-terminal Signal Peptide
ERIKDLTTIQGVRSNQLIGYGLVVGLDGTGDQTTQTPFTVQSIVSMMQQMGINLPSGTNLQLRNVAAVMVTGNLPPFAQPGQPMDVTVSSMGNARSLRGGTLLMTPLKGADNQVYAMAQGNLVIGGAGAGASGTSTQINHLGAGRISAGAIVERAVPSQLTETSTIRLELKEADFSTASMVVDAINKRFGNGTATPLDGRVIQVQPPMDINRIAFIGNLENLDVKPSQGPAKVILNARTGSVVMNQAVTLDDCAISHGNLSVVINTAPAISQPGPFSGGQTVATQVSQVEINKEPGQVIKLDKGTSLADVVKALNAIGATPQDLVAILQAMKAAGSLRADLEII

SEQ ID NO: 209 - protein, 24 aa, Artificial Sequence
  N-terminal Signal Peptide
MTLTRPLALISALAALILALPADA

SEQ ID NO: 210 - protein, 100 aa, Artificial Sequence
  N-terminal Signal Peptide
DGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGD

SEQ ID NO: 211 - protein, 16 aa, Artificial Sequence
  N-terminal Signal Peptide
MKLLTSFVLIGALAFA

SEQ ID NO: 212 - protein, 116 aa, Artificial Sequence
  N-terminal Signal Peptide
MKLLTSFVLIGALAFADGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGD